SEQUENCE LISTING (7 sequences)

SEQ ID NO: 1 - DNA, 23 bp, Artificial
  synthetic
ctgaaagcatgatccgggacgtg

SEQ ID NO: 2 - DNA, 23 bp, Artificial
  synthetic
tgaccttggtctggtaggagacg

SEQ ID NO: 3 - DNA, 21 bp, Artificial
  synthetic
gtcactgtcagaggctaagtt

SEQ ID NO: 4 - DNA, 20 bp, Artificial
  synthetic
gcatcgacaacaccacggtc

SEQ ID NO: 5 - DNA, 24 bp, Artificial
  synthetic
gtccatgtcactgatctttctggc

SEQ ID NO: 6 - DNA, 24 bp, Artificial
  synthetic
gtttgatgtgtagggcggtgatac

SEQ ID NO: 7 - protein, 80 aa, Artificial
  synthetic
HRETEGEEQAQLLHVSSPNLASDSDLSRRSSSTISKSEYMEIEEDMNNSIAHYRQANIRTGNCTTADQNCVNKSKLLTDV

(19) United States
(12) Patent Application Publication
(10) Pub. No.: US 2009/0022739 A1
(43) Pub. Date: Jan. 22, 2009

(54) MODULATING THE KV1.1 VOLTAGE-GATED POTASSIUM CHANNEL IN T-CELLS FOR REGULATING THE SYNTHESIS AND SECRETION OF TUMOR NECROSIS FACTOR ALPHA (tnf-ALPHA) AND TREATING HUMAN DISEASE OR INJURIES MEDIATED BY DETRIMENTALLY HIGH OR LOW LEVELS OF TNF-ALPHA

(76) Inventor: Mia Levite, Savyon (IL)

Correspondence Address: BROWDY AND NEIMARK, P.L.L.C.;624 NINTH STREET, NW, SUITE 300, WASHINGTON, DC, 20001-5303, US

(21) Appl. No.: 11/997,776

(22) PCT Filed: Aug. 3, 2006
(86) PCT No.: PCT/US06/30361

§ 371 Date: Feb. 4, 2008

Related U.S. Application Data
(60) Provisional application No. 60/704,728, filed on Aug. 3, 2005.

(51) Int. Cl. A61K 39/395; C12P 21/00; A61K 35/28; A61K 38/16; A61P 37/02; A61P 43/00

(52) U.S. Cl. 424/173.1; 435/70.4; 424/93.71; 514/12

(57) ABSTRACT

Blocking the voltage-gated potassium channel Kv1.1 of T-cells causes the robust and exclusive production of TNF-α, and thus can be used for eradication of cancer, improved eradication of infectious organisms, increased permeability of blood vessels and the blood brain barriers to given molecules and cells, and improved neuronal features, regeneration function and development. Blocking the voltage-gated potassium channel Kv1.1 of T-cells causes the robust and exclusive production of TNF-α. Similarly, unblocking of a blocked Kv1.1 channel or opening of a Kv1.1 channel will prevent the T-cells from producing and secreting excess amounts of TNF-α, thus being useful in the treatment of conditions such as rheumatoid arthritis and for treating neurological diseases associated with defected functioning and/or pathological block of the Kv1.1 channel, among them PNH associated with Kv1 Abs; Encephalitis associated with Kv1 Abs; and Episodic-ataxia type 1 (EA-1), in all of which the T-cell blocked Kv1.1 channel may secrete excess TNFa and thereby contribute to the pathology. Blocking of the Kv1.1 channel may be achieved in vivo or ex vivo by contact with a selective Kv1.1 channel blocking molecule such as Dendrotoxin-K or a selective monoclonal antibody against the Kv1.1 channel. Preventing the Kv1.1. block would be achieved by Kv1.1 openers, or by molecules that would prevent the closure of the Kv1.1 channel.

### BACKGROUND OF THE INVENTION

Tumor necrosis factor alpha (TNF-α), isolated 30 years ago, is a multifunctional cytokine playing a key role in apoptosis and cell survival, as well as in inflammation and immunity. Although named for its antitumor properties, TNF has been implicated in a wide spectrum of other diseases. The current use of TNF in cancer is in the regional treatment of locally advanced soft tissue sarcomas and metastatic melanomas and other irresectable tumors of any histology to avoid amputation of the limb. It has been demonstrated in the isolated limb perfusion setting that TNF-α acts synergistically with cytostatic drugs. The interaction of TNF-α with TNF receptor 1 and receptor 2 (TNFR-1, TNFR-2) activates several signal transduction pathways, leading to the diverse functions of TNF-α. The signaling molecules of TNFR1 have been elucidated quite well, but regulation of the signaling remains unclear. Besides these molecular insights, laboratory experiments in the past decade have shed light upon TNF-α action during tumor treatment. Besides extravasation of erythrocytes and lymphocytes, leading to hemorrhagic necrosis, TNF-α targets the tumor-associated vasculature (TAV) by inducing hyperpermeability and destruction of the vascular lining. This results in an immediate effect of selective accumulation of cytostatic drugs inside the tumor and a late effect of destruction of the tumor vasculature. A recently published review (see van Horssen et al (2006)), covers TNF-α from the molecule to the clinic, and provide an overview of the use of TNF-α in cancer starting with molecular insights into TNFR-1 signaling and cellular mechanisms of the antitumor activities of TNF-α and ending with clinical response. In addition, possible factors modulating TNF-α actions are discussed.

TNF-α was isolated in 1975 from the serum of mice treated with bacterial endotoxin as the active component of “Coley's toxin” and was shown to induce hemorrhagic necrosis of mice tumors. It was almost a century ago that William Coley, a surgeon from New York, observed high fever and tumor necrosis in some cancer patients treated with his bacterial filtrate (“Coley's mixed toxins”). A decade after its isolation, TNF-α was also characterized as “cachectin” and as T-lymphocyte differentiation factor. In 1984, the human TNF-α gene was cloned, and a range of clinical experiments were set up, leading to a license from the European Agency for the Evaluation of Medicinal Products (EMEA) for the treatment of limb-threatening STS in an isolated perfusion setting (for all the above see van Horssen et al (2006) and papers cited therein).

By activating its two high affinity receptors, TNFR1 (p55) and TNFR2 (p75), expressed on virtually all cell types except erythrocytes, TNF-α plays a major role in a kaleidoscope of conditions including acquired and innate immunity, induction of inflammation required for combating foreign invaders such as viruses and bacteria, eradication of tumors, promotion of the regeneration and proliferation of oligodendrocytes, induction of nerve remyelination and many others.

T-cells (also called T-lymphocytes) are a major source for TNF-α. The primary stimulus known to induce the secretion of TNF-α from T-cells is the engagement of the T cell receptor (TCR), alike occurring upon encounter of T-cells with an antigen presented to them by antigen presenting cells, in the context of self-major histocompatibility complex molecules. Such “classical” TCR-activation drives T-cells into a robust but non selective cytokine secretion (i.e., simultaneous secretion of all the cytokines that can be secreted by these cells).

TNF-α plays a dual role in human physiology and pathology: on the one hand, TNF-α is crucially needed to fight disease and heal; but on the other hand, excess TNF-α is detrimental as it leads to diseases and tissue destruction.

It is generally assumed that TNF-α primary function in activating the innate immune response is beneficial and crucial for protecting the body from various types of foreign invaders, and for eradication of cancer cells. TNF-α also substantially increases the permeability of blood vessels and of the blood brain barrier (BBB) to various molecules, and augments the rolling and adhesion of leukocytes which is required for their migration and penetration into tissues. Thus, it is absolutely clear that lack or insufficient TNF-α may have wide pathological consequences, among them immunocompetence and inability to cope with infectious organisms, and to eradicate cancer etc.

Based on the ability of TNF-α to kill cancer cells, it is currently, used in cancer treatment in the isolated limb perfusion (ILP) setting for soft tissue sarcoma (STS), irresectable tumors of various histological types, and melanoma in transit metastases confined to the limb (see van Horssen et al (2006) and papers cited therein).

Table 1 in van Horssen et al (2006) presents an overview of the multicenter trials in Europe that led to the approval of TNF-α by the EMEA in 1998 for its application in ILP for the treatment of high-grade (Wallach et al, 1999; Mayan, 2002) STS. In these multicenter trials, an overall response rate of 76% and a median limb salvage rate of 82% were observed. Moreover, this table lists the largest single-center studies in STS that confirm the results of the multicenter experience. Strikingly consistent major response rates were observed, with a median of 76% (range, 58%-91%), and with a median limb salvage rate of 84% (range, 58%-97%). TNF-α-based ILP now is performed in 35 cancer centers in Europe with national referral patterns for limb salvage. ILP with melphalan alone for melanoma in-transit metastases is reported in the literature to result in about a 50% complete response (CR) rate and an 80% overall response rate (see van Horssen et al (2006) and papers cited therein). The introduction of TNF-α in this setting was reported to increase CR rates to 70%-90% and overall response rates to 95%-100%. These results are summarized in Table 2 in van Horssen et al (2006).

On the other hand, it is also clear and documented in numerous studies that an inappropriate or over-production of TNF-α in humans lead to uncontrolled detrimental inflammation, tissue destruction and organ injury. Such over-production of TNF-α occurs in various autoimmune diseases, such as rheumatoid arthritis and multiple sclerosis, inflammatory diseases, such as inflammatory bowel disease, and other pathological conditions, among them, for example, ankylosing spondylitis and stroke.

In the central nervous system (CNS), TNF-α also plays a dual role: on the one hand TNF-α is beneficial and needed for proliferation and regeneration of oligodendrocytes, neuroprotection, induction of nerve remyelination and various others important processes. On the other hand, excess endogenous TNF-α contributes to neuronal injury and neurodegeneration. Increasing attention is being paid to the role of inflammatory and immune molecules in the modulation of central nervous system (CNS) function. Tumor necrosis factor-alpha (TNF-α) is a pro-inflammatory cytokine, the receptors for which are expressed on neurons and glial cells throughout the CNS. Through the action of its two receptors, it has a broad range of actions on neurones which may be either neuroprotective or neurotoxic. It plays a facilitatory role in glutamate excitotoxicity, both directly and indirectly by inhibiting glial glutamate transporters on astrocytes. Additionally, TNF-α has direct effects on glutamate transmission, for example increasing expression of AMPA receptors on synapses. TNF-α also plays a role in synaptic plasticity, inhibiting long term potentiation (LTP), a process dependant on p38 MAP kinase.

It is clear from the above that in the specific pathologies characterized by a lack of or insufficient TNF-α, i.e., a lack of or insufficient immune competence or other TNFα-mediated beneficial effects, the finding of novel methods to exclusively boost TNFα production, expression and secretion, without augmenting any other cytokines, would be of substantial therapeutic value.

The following represent examples of pathological conditions whereby augmenting TNF-α and its subsequent effects on target cells can be a therapeutic strategy:

a. cancer, in which failure to kill or remove the tumor leads to death (see van Horssen et al (2006), a recent review of TNF-α antitumor effects and clinical utility, the entire contents of which are hereby incorporated herein by reference);

b. chronic or acute immunodeficiency, resulting in inability to combat foreign invaders alike viruses and bacteria, which may lead to death; and

c. disease or injury-associated lack or insufficient neuronal regeneration that may culminate in loss of crucial neuronal functions and endanger life.

It is also clear from the preceding introduction that in specific pathologies characterized by excess and detrimental TNF-α, the finding of novel methods to arrest TNF-α production, expression and secretion would be of substantial therapeutic value.

The following are examples of pathological conditions whereby arresting or decreasing TNFα and its subsequent effects on target cells can be a therapeutic strategy: rheumatoid arthritis, psoriatic arthritis, type 1 diabetes mellitus, Crohn's Disease, psoriasis, ulcerative colitis, ankylosing spondylitis, sarcoidosis and post head injury.

### SUMMARY OF THE INVENTION

In studies serving as the basis of this patent application, the present inventors have found that the selective closure of the voltage-gated potassium channel Kv1.1 in T-cells is by itself sufficient (i.e., in the absence of any additional molecules) to cause a dramatic and exclusive synthesis and secretion of TNF-α, while not affecting any of the other cytokines tested, i.e., interferon gamma, interleukin-4 and interleukin-10.

On the basis of these findings, it has been determined that the closure or blockade of the voltage-gated potassium channel Kv1.1 in T-cells will achieve the rapid, robust and selective synthesis and secretion of TNF-α by T-cells in the situations where such TNF-α-mediated effects are of therapeutic value.

The closure of the voltage-gated potassium channel Kv1.1 in T-cells is achieved herein using highly selective Kv1.1 channel blockers, such as Dendrotoxin-K (DTX-K), or specific anti-Kv1.1 antibodies, or by any other methods that lead to the selective blockade of the T-cell Kv1.1 channels, either extracellularly or intracellularly by means of blockage of downstream pathways.

Accordingly, an object of the present invention is to provide a method for blocking the Kv1.1 channel in T-cells in human or animal subjects suffering from pathologies linked to the lack of TNF-α in order to combat the disease or the injury, by inducing T-cells to produce and secrete high doses of TNF-α.

A further object of the present invention is to provide such a method in which the molecule used to block the voltage-gated-potassium channel Kv1.1 in T-cells is a selective Kv1.1 ion channel blocker.

A further object of the present invention is to provide a method for blocking the Kv1.1 channel in T-cells in human or animal subjects that can benefit from augmented and exclusive secretion of TNF-α by T-cells in order to combat the disease or the injury, by inducing T-cells to produce and secrete high doses of TNF-α.

Another object of the present invention is to provide such a method in which the molecule used to block the voltage-gated potassium channel Kv1.1 in T-cells is a specific anti-Kv1.1 antibody.

Yet another object of the present invention is to provide such a method in which the molecule used to block the voltage-gated potassium channel Kv1.1 in T-cells is a molecule able to block the downstream pathways of the Kv1.1 channel in T-cells.

A still further object of the present invention is to provide such a method in which the molecule used to block the voltage-gated potassium channel Kv1.1 in T-cells is administered to T-cells ex vivo prior to their inoculation back into the body of a human or animal patient.

A further object of the present invention is to provide such a method in which the molecule used to block the voltage-gated potassium channel Kv1.1 in T-cells is administered in vivo, i.e., into the body of a human or animal patient by intravenous, subcutaneous, intraperitoneal, intratumoral, intrathecal or intracranial injections, or by oral administration or via transdermal ointments.

Yet a further object of the present invention is to provide such a method in which the molecule used to block the voltage-gated potassium channel Kv1.1 in T-cells is administered into the body of human or animal subjects using an implantable drug-delivery pump.

Another object of the present invention is to provide such a method in which the human or animal subjects suffer from cancer and for whom the exposure of their own T-cells, either ex vivo or in vivo, to molecules that block the T-cell Kv1.1 channels, will trigger the robust synthesis of secretion of TNF-α, leading to the direct killing of the cancer by TNF-α.

In a preferred embodiment, the human or animal subjects suffer from cancer and for whom the exposure of their own T-cells, either ex vivo or in vivo, to molecules that block the T-cell Kv1.1 channels, will trigger the robust synthesis and secretion of TNF-α in order to enhance the T-cell immunotherapy of cancer by increasing the penetration of the T-cells into the tumor bearing organs, and by creating a beneficial inflammatory milieu to improve the recruitment of tumor-destructing cells.

In another preferred embodiment, the human or animal subjects suffer from immunodeficiency.

In yet another preferred embodiment, the human or animal patients suffer from deficiency in neuronal regeneration after neuronal injury or neurological disease.

In another preferred embodiment of the present invention, the procedure to obtain robust synthesis and secretion of TNF-α serves to achieve TNF-α-induced augmentation animal patient in vivo by intravenous, subcutaneous, intraperitoneal, intratumoral, intrathecal, or intracranial injections, or by oral administration or via transdermal ointments.

In the second category of diseases, the detrimental excess of TNF-α is overcome in accordance with this invention, by preventing TNF-α production and secretion from T-cells with molecules that do not permit the closure/block of the Kv1.1 channels induced by putative endogenous disease-associated Kv1.1 blockers. If the latter (i.e., putative endogenous disease-associated Kv1.1 blockers) are the cause for chronic TNF-α production and secretion in T-cells, preventing them from blocking the Kv1.1 channel, for example, by Kv1.1 channel openers, will also prevent them from triggering the robust TNFα elevation. Molecules that open the channel and thus prevent TNF-α production may be either extracellular agonists or molecules that impair the intracellular voltage sensing element that signals blockage of the channel. An example of such an extracellular agonist is a monoclonal antibody raised against the Kv1.1 molecule and screened for agonistic activity, in a conventional manner.

### BRIEF DESCRIPTION OF THE DRAWINGS

FIG. 1A is a graph showing the fold increase of TNF-α for each tested ion channel blocker.

FIG. 1B is a graph showing the fold increase of TNF-α as a function of dose.

FIGS. 1C-H are graphs for six different individuals, respectively, each showing the amount of DTX-K-induced TNF-α secretion.

FIGS. 2A-H show the levels of various cytokines secreted by normal human T-cells exposed to either DTX-K (FIGS. 2A-D) or to anti-CD3 and anti-CD28 mAbs (FIGS. 2E-H). Fold increases of TNF-α secretion is shown in FIGS. 2A and 2E, IFNγ (FIGS. 2B and F), IL-10 (FIGS. 2C and G) and IL-4 (FIGS. 2D and H).

FIGS. 2I and J are graphs showing the fold increase of TNF-α secretion as a function of time in two different individuals, respectively.

FIG. 2K is a graph showing TNFα secretion for T-cells that are either untreated, treated with DTX-K alone, or first pretreated with either Bay-K or NPPB.

In FIG. 3A, the upper bands show TNF-α (expected product size: 494 bp), and the lower bands show S14 (expected product size: 166 bp), following exposure of normal peripheral human T-cells to DTX-K. The bars at the bottom represent fold increase TNF-α mRNA calculated following densitometric analysis and normalization of the corresponding RT-PCR bands of TNF-α/S14. NC=negative control (no cDNA).

FIG. 3B shows the amount of NF-κB bound to nuclear NF-κB-binding sequences in nuclear extracts prepared from normal human T-cells pre-exposed to 100 nM DTX-K.

FIG. 3C shows double immunofluorescence photographs in which normal human T-cells were either left untreated or treated with 100 nM DTX-K for 7, 15, 30 and 60 minutes (see DTX-K treatment time above each respective column). Representative photographs at ×100 magnification are shown for NF-κB p50 green fluorescence (upper panel), nuclei blue fluorescence (middle panel), and overlay of both (lower panel). The overall sizes of the studied T-cells (bright field) and nucleus (Hoechst) are shown in the left column.

FIG. 4A shows the results of Kv1.1 specific RT-PCR performed using Kv1.1-specific primers (upper band; expected product size: 709 bp). Control RT-PCR was performed in parallel in the same PCR tube using ribosomal S14 primers (lower band; expected product size: 166 bp).

FIG. 4B shows the results of a FACSort after T-cells were stained either with a rabbit anti-Kv1.1 polyclonal Ab (FIG. 4B, solid bold line), or, as isotype control non-specific staining, with normal rabbit IgG (FIG. 4B, dashed line), and then with FITC-conjugated anti-rabbit IgG.

FIGS. 4C-E show results in three different individuals, respectively, of the mean TNF-α secretion by T-cells of each individual after such T-cells were incubated with a commercial rabbit anti-Kv1.1 polyclonal Ab.

FIGS. 4F and G show the levels of TNF-α (FIG. 4F) or IFN-γ (FIG. 4G) secreted into the medium by normal peripheral human T-cells were exposed to sera of either healthy control individuals (group A), Kv1 Ab-positive PHN patients (group B), or Kv1 Ab-negative PHN patients (group C).

FIGS. 5A-F show the results after freshly-purified normal human T-cells were exposed to either PBS or to 100 nM DTX-K for 24 hours, washed, fluorescently-labeled and then injected into SCID mice. The graphs show the total number of cells, as well as only of the resident SCID cells (non-fluorescent) and only of the injected human T-cells (fluorescent) present in single cell suspensions of liver, bone marrow, spleen and kidney, as well as in blood. FIG. 5A shows the mean±SEM number of non-fluorescent SCID host cells in the liver and FIG. 5B shows the mean±SEM of the fluorescently-labeled human T-cells in the liver. In regards to the other organs, only the number of non-fluorescent SCID host cells is shown: bone marrow (FIG. 5C), blood (FIG. 5D), spleen (FIG. 5E) and kidney (FIG. 5F).

FIG. 5G shows immunohistochemistry of brain slices from SCID mice injected with human T-cells preincubated ex vivo with DTX-K, for the detection of the infused human T-cells. A representative photograph at ×40 magnification is presented, showing that 24 hours following injection of DTX-K-treated human T-cells into SCID mice, human CD3-positive cells are detected within the cortex.

### DETAILED DESCRIPTION OF PREFERRED EMBODIMENTS

The present invention is based on the discovery that selective block of Kv1.1 voltage-gated potassium channels in normal human T-cells induces by itself robust and exclusive elevation of TNF-α protein secretion and mRNA. Dramatic TNF-α secretion was induced by the highly selective Kv1.1 blocker Dendrotoxin-K (but not by nine other K⁺, Na⁺, Ca²⁺ and Cl⁻ blockers); by commercial anti-Kv1.1 Abs; and, importantly, by sera from Peripheral Nerve Hyperexcitability patients harboring Kv1.1 Abs. Kv1.1 block triggered only TNF-α secretion, while “classical” TCR-activation elevated simultaneous and non-exclusive secretion of TNF-α, IFN-γ, IL-4 and IL-10. Kv1.1 block also induced nuclear translocation of NF-κB.

Kv1.1 protein and mRNA expression in human T-cells was demonstrated herein by RT-PCR and flow cytometry. Injection into SCID mice of human T-cells pretreated ex vivo with Dendrotoxin-K and thus over-secreting TNF-α, caused in vivo recruitment of host cells into the liver, like that which has been reported after TNF-α injection to the brain. Nothing known thus far could predict these findings. TNF-α elevation induced by Kv1.1 block may therefore be used therapeutically, such as by boosting TNF-α in certain diseases (e.g., cancer and immunodeficiencies) and arresting it in others (e.g., rheumatoid arthritis, Crohn's and other autoimmune/inflammatory diseases).

The present invention establishes that a selective block of one type of ion channel in human T-cells can, by itself, trigger the exclusive production and secretion of one type of cytokine, in this case, TNF-α. Such a mechanism contrasts starkly with both “classical” TCR-activation and non-specific T-cell stimulation by various mitogens (e.g., phorbol esters) that characteristically drive the simultaneous production of all T-cell cytokines. The discovery of a mechanism that exclusively and potently elevates a single cytokine while not increasing the level of other Th cytokines has important clinical implications, especially as the cytokine is TNF-α (Dinarelo et al, 2002; Wallach et al, 1999), since this cytokine is crucial in the pathology of many disorders, in which it is either in excess or in insufficient levels.

Indeed, on the one hand, TNF-α plays a cardinal and beneficial role in health. It activates the innate immune response and induces essential inflammation, which protects the body from environmental attack (Dinarelo et al, 2002). In addition, TNFα causes necrosis of tumors, augments rolling and adhesion of leukocytes, increases the permeability of blood vessels and the blood brain barrier (BBB) to various molecules (Mayan 2002, van Horssen et al 2006), induces proliferation and regeneration of oligodendrocytes and promotes nerve remyelination (Arnett et al, 2001; Finsen et al, 2002), recruits cells into specific tissues and induces many other important functions (Dinarelo et al, 2002; Wallach et al, 1999).

On the other hand, excess TNF-α or its unregulated production is usually highly detrimental and can contribute to severe and damaging inflammation leading to tissue destruction, which subsequently contributes to various severe autoimmune diseases, among them rheumatoid arthritis and multiple sclerosis, inflammatory diseases, among them inflammatory bowel disease, and various types of other pathological conditions, as diverse as injury, ankylosing spondylitis, stroke and narcolepsy. Interestingly, while there is an ever growing list of clinical indications for therapies that block TNF-α signaling once it is in excess, such as anti-TNF-α monoclonal Abs and soluble TNF-α receptor, preventing or blocking TNF-α elevation (i.e., before it reaches excess detrimental levels) has not been achieved yet. Furthermore, the mechanisms responsible for the chronic production of TNF-α in human diseases are still poorly understood, and a mechanism that selectively promotes TNF-α elevation, while not affecting other cytokines, has not been revealed yet.

Prompted by the known properties of specific ion channels to influence and dictate key cellular responses in various cells types including T-cells (DeCoursey et al, 1984; Deutsch et al, 1986, Lewis et al, 1995; Lewis et al, 1988; Rader et al, 1996; Freedman et al, 1995; Koo et al, 1997; Lin et al, 1993; Ishida et al, 1993; Jansen et al, 1999; Kotturi et al, 2003; Phipps et al, 1996; Verheugen et al, 1997), and the previous demonstration from the laboratory of the present inventors that gating of Kv1.3 voltage-gated potassium channels can by itself trigger key T-cell function (Levite et al, 2000), the present invention is based on the study of whether the mere blocking of specific potassium (K+), sodium (Na⁺), calcium (Ca²⁺) or chloride (Cl⁻) channels in normal human T-cells could trigger selective cytokine secretion, especially of TNF-α. The results demonstrate a novel mechanism for the robust, exclusive and prolonged elevation of TNF-α secretion by the selective blocking of Kv1.1 subunit containing voltage-gated K⁺ channels in normal human T-cells.

The present findings reveal a novel and unexpected mechanism whereby selective block of the Kv1.1 subunit containing voltage-gated K⁺ channels in normal peripheral human T-cells triggers marked, exclusive and prolonged elevation of TNF-α secretion and mRNA levels. To the best of our knowledge, nothing known thus far about voltage-gated K⁺ channels in general and Kv1.1 in particular, or about TNF-α, could have predicted the present findings of a direct functional association between Kv1.1 and TNF-α. Furthermore, the present findings reveal a novel mechanism for isolated elevation of TNF-α, without elevating other Th cytokines.

In the following paragraphs, there is discussed: a) the present findings in further detail; b) the Kv1.1 voltage-gated K⁺ channel and other members of the Kv family primarily in T-cells, and the unique features of DTX-K; c) PNH, the human neurological disease associated with Kv1 block; and d) the use of T-cell Kv1.1 as a novel clinical target to either boost or arrest TNF-α in different human diseases.

### The Present Findings in Further Detail

It has been found that both the highly selective Kv1.1 blocker DTX-K (Harvey, 2001) and specific commercial anti-Kv1.1 Abs induced dramatic elevation of T-cell TNF-α. By contrast, exposing T-cells to nine other blockers of K⁺, Na⁺, Ca²⁺, Cl⁻ ion channels or glutamate/AMPA ionotropic receptors expressed in T-cells (Ganor et al, 2003), had no such effect. The two Kv1 blockers KTX and MgTX, which are not specific for Kv1.1 but can somewhat affect Kv1.1 subunit containing voltage-gated K⁺ channels, also triggered TNFα from human T-cells, but to a much lower extent, in line with their lower affinity and specificity to the Kv1.1 channel subunit.

While KTX and MgTX are not as specific to the Kv1.1 channel as DTX-K, they are sufficiently specific to be usable in accordance with the present invention. The present invention does not comprehend totally non-specific T-cell activators, such as phorbol esters, as exemplified by PMA. However, the term “selective” as used herein is intended to include Kv1.1 blockers that may also have some activity on other Kv channels, as long as T-cell cytokines are not indiscriminately produced, as is the case, for example, with phorbol esters, such as PMA. The greater the selectivity for Kv1.1 channels, the greater the preference for such a molecule. Thus DTX-K is the most preferred such molecule at the present time. However, although they may not produce as much TNF-α as DTX-K and may not be as specifically selective to Kv1.1 as DTX-K, KTX and MgTX can also be used in accordance with the present invention. Thus, any of the following fully selective and less selective blockers of the Dv1.1 voltage-gated ion channel may be used in accordance with the present invention, as are any other molecules that block the Kv1.1 channel and are at least substantially selective to Kv channels:

A) Dendrotoxins
1) Toxin K, termed also Dendrotoxin-K (DTX-K), from the black mamba Dendroaspis polylepis, preferentially blocks Kv1.1 channels and is active at picomolar concentrations.
2) Alpha-dendrotoxin from green mamba Dendroaspis angusticeps block cloned Kv1.1, Kv1.2 and Kv1.6 channels in the low nanomolar range.
3) Toxin I, termed also Dendrotoxin-I (DTX-I), from the black mamba Dendroaspis polylepis block cloned Kv1.1, Kv1.2 and Kv1.6 channels in the low nanomolar range.

B) Conotoxins: From the venom of the cone snail Conus virgo, a peptide named ViTx (virgo-toxin) was isolated which blocks K⁺-channels of the Kv1.1 and Kv1.3, but not of Kv1.2 type (Kauferstein et al, 2003).

C) Maurotoxins: Both natural and synthetic maurotoxins blocked the Kv1.1, Kv1.2, and Kv1.3 channels expressed in Xenopus oocytes with almost identical half-effects (IC50) in the range of 40, 0.8 and 150 nM (Rochat et al, 1998).

D) Agitoxins
1) rAgitoxin-2 Leiurus q. hebraeus, blocks Kv1.1, Kv1.3, Kv1.6. 50 pM-10 nM. (Alomone Labs web site: www.alomone.com/p_products).
2) rAgitoxin-3 Leiurus q. hebraeus, blocks Kv1.1, Kv1.3, Kv1.6. 50 pM-10 nM. (Alomone Labs web site: www.alomone.com/p_products).

E) rHongotoxin-1 Centruroides limbatus, blocks Kv1.1, Kv1.2 and Kv1.3 0.1-0.2 pM.

F) Pi4, a 38-amino acid long peptide from the venom of the scorpion Pandinus imperator, blocks completely and reversibly Shaker B K⁺-channels (a Kv1.1 sub-family type of channel) at 100 nM concentration (Olamendi-Portugal 1998)

G) Kaliotoxin (KTX): KTX blocks some Ca²⁺ activated and voltage-gated K⁺ channels. Bath application of KTX to Xenopus laevis oocytes blocked recombinant Kv1.3 and Kv1.1 channels potently and Kv1.2 channels less potently, with respective K(d) values of 0.1, 1.5, and 25 nM (Mourre et al, 1999)

H) BTK-2: A new inhibitor of the Kv1.1 potassium channel purified from Indian scorpion Buthus tamulus (Dhawan et al, 2003).

I) A novel Kv1.1 potassium channel blocking toxin: A novel Kv1.1 potassium channel blocking toxin from the venom of Palamneus gravimanus (Indian black scorpion) (More et al, 2005).

J) rMargatoxin: MgTX K⁺ channels, which blocks primarily the Kv1.3 voltage-gated K⁺ channels, but can also affect the Kv1.1.

K) Stichodactyla helianthus peptide (ShK), isolated from S. helianthus venom and a known high-affinity blocker of Kv1.1 and Kv1.3 channels.

The antibodies against Kv1.1 used in the present experiments are commercially available from Alamone Labs Ltd., Jerusalem, Israel, product no. APC-009. See the datasheet available at alomone.com/System/UpLoadFiles/DGallery/Docs/apc-009_AN-05.pdf. This antibody is a rabbit polyclonal antibody raised using GST fusion protein with sequence HRETE GEEQA QLLHV SSPNL ASDSD LSRRS SSTIS KSEYM EIEED MNNSI AHYRQ ANIRT GNCTT ADQNC VNKSK LLTDV (SEQ ID NO:7), corresponding to residues 416-495 of mouse KV1.1 (GenBank Accession P16388). 76 of the 80 residues of this epitope are conserved in the corresponding human protein. Any other antibody raised against an epitope of Kv1.1, preferably human Kv1.1, whether polyclonal or monoclonal, may be used for the purpose of the present invention as long as they have the capability of blocking the Kv1.1 channel. The term “antibody” as used in the present specification and claims, is intended to comprehend polyclonal or monoclonal antibodies, as well as genetically engineered antibodies, such as humanized antibodies, single-chain antibodies, and antigen-binding antibody fragments, such as CDRs, Fab, F(ab)2, etc.

When producing monoclonal antibodies against the Kv1.1 channel, they may be screened for their ability to either block (antagonize) or open (agonize) the channel. Antibodies that are antagonists can be used to treat conditions in which increased production of TNF-α is desired and antibodies that are agonists can be used to treat diseases where excess production of TNF-α needs to be prevented.

In line with the known role of Ca²⁺ in TNF-α transcription (Lobo et al, 1999), it was found that TNF-α induction by Kv1.1 block requires Ca²⁺ fluxes, as R-(+)-Bay K 8644, which is a highly selective blocker of Ca²⁺ channels in T-cells (Kotturi et al, 2003), prevented this effect. Strikingly, blocking the Kv1.1 channels, while causing sharp elevation of TNFα, did not elevate IFN-γ, IL-4 and IL-10, and as such differed markedly from the “classical” TCR-activation, which lead to a robust yet non-selective and concomitant elevation of all these Th cytokines.

The TNF-α elevation induced in human T-cells by the selective block of the Kv1.1 channel had functional consequences in vivo, as the injection into SCID mice of DTX-K treated normal human T-cells which over expressed TNF-α, doubled the number of mouse resident cells present exclusively in the liver, similar to the results shown in a recent study to occur following injection of TNF-α into the brain (Campbell et al, 2005).

The Kv1.1 voltage-gated K+ channel and other members of its family, and the unique features of DTX-K: The Kv1.1 channel belongs to a family of voltage-gated K⁺ channels, of which several members are expressed by and play an important functional role in T-cells (DeCoursey et al, 1984; Deutsch et al, 1986; Lewis et al, 1995; Lewis et al, 1998; Freedman et al, 1995; Koo et al, 1997; Lin et al, 1993; Levite et al, 2000; Liu et al, 2002; Beeton et al, 2001; Leonard et al, 1992; Levite 2001). These channels are different from Ca²⁺-activated K⁺ channels, which are also expressed by and important for T-cell function (Rader et al, 1996; Jensen et al, 1999; Vreheugen et al, 1997). The grouping and characteristic features of the different types of voltage-gated K⁺ channels are discussed by several studies (see Grissmer et al, 1994; Mathis et al, 1998). In general, inhibition of the voltage-gated K⁺ channels causes depolarization (Leonard et al, 1992) (i.e., a shift towards less negative membrane potential), which in turn affects and allows various key cellular features and functions to take place (Levite et al, 2000; Leonard et al, 1992; Levite, 2001). In neurons, one major role of voltage-gated channels is to control membrane excitability (Ruteckie, 1992; Smart et al, 1998). This includes limiting the duration of the action potential and repetitive neuronal firing, and stabilizing the membrane potential.

Specific voltage-gated K⁺ channels in T-cells were shown in previous studies to activate β1 integrins, and trigger subsequent T-cell adhesion to ECM components alike fibronectin (Levite et al, 2000), and to regulate the membrane potential (Levite et al, 2000), calcium influxes (Grinstein et al, 1990; Lin et al, 1993; Verheugen et al, 1997), proliferation (Freedman et al, 1992; Leonard et al, 1992), IL-2 production (Liu et al, 2002, Freedman et al, 1992), apoptosis (Bortner et al, 1999; maeno et al, 2000; Vu et al, 2001), and the cell volume (Grinstein et al 1990).

Thus, as a whole, Kv channels regulate key physiological T-cell functions, and their inappropriate function inhibits TCR-activation and prevents crucial T-cell mediated immune responses. For example, Liu et al (2002) showed, by combination of electrophysiological, pharmacological and RT-PCR methodologies, that naïve murine CD4⁺ T cells express Kv1.1, Kv1.2, Kv1.3 and Kv1.6 currents, and that all these Kv channels are required for maximal TCR-induced (by anti-CD3 and anti-CD28) IL-2 secretion by such naïve cells (Liu et al, 2002).

In contrast, mouse TCR-activated effector T-cells, express only Kv1.3 currents (which are +6 fold higher than in naïve cells), and neither Kv1.3 nor any other Kv contribute significantly to IL-2, IL-4 or IFN-γ production, calcium signaling or the membrane potential of these activated T-cells (Liu et al, 2002). The observations made in this latter study indicated that in peripheral murine CD4⁺ T-cells, Kv currents change qualitatively and quantitatively as a reflection of the differentiation state, the strength of TCR-activation and the presence or absence of costimulatory molecules (Liu et al, 2002). Native voltage-gated K⁺ channels are heteromultimers, composing of a combination of various Kv subtypes, and different heteromultimeric channels could have dramatically different channel functions, compared to the parent channels. Different Kv1.1 containing heterodimers also have different sensitivity to different blockers. For example, the presence of Kv1.2 in the Kv1.1 containing heterodimer makes the whole array much less sensitive to TEA. Previous studies showed that DTX-K is unique in its strong and highly specific interaction with the Kv1.1 subunits, and that it interacts with these channels through residues in its N-terminus (K3, K6) and β-turn (K25, K26) (Wang et al). The highly unique mode of action of DTX-K may account for the DTX-K induced TNF-α elevation (in a yet unexplored mechanism), while the mode of action of less specific Kv1 blockers lead to a much less pronounced effect, if at all.

Different studies, testing for Kv1.1 currents in human and mouse T-cells, reached different conclusions (Freedman et al, 1995; Cahalan et al, 1985; Liu et al, 2002). The present results show, by specific Kv1.1 RT-PCR and flow cytometry, that a substantial proportion of normal peripheral human T-cells clearly express the Kv1.1 mRNA and membranal protein, and reveal a new facet of this protein. Yet, our findings of course do not prove the existence of functional (i.e., electrophysiologically active) Kv1.1 currents in normal resting peripheral human T-cells. Evidence that other members of the voltage-gated K⁺ channel family, primarily the Kv1.3 channels, play a critical role in T-cell activation and function is suggested by many recent studies (DeCoursy et al, 1984; Deutsch et al, 1986; Lewis et al, 1995; Lewis et al, 1988; Freedman et al, 1995; Koo et al, 1997; Lin et al, 1993; Leite et al, 2000; Liu et al, 2002; Beeton et al, 2001; Leonard et al, 1992; Rus et al, 2005). Furthermore, the laboratory of the present inventors previously demonstrated that the gating of a single member of the voltage-gated K⁺ channels in normal human T-cells, the Kv1.3, can on its own trigger β1 integrin activation and T-cell adhesion, and that the Kv1.3 channel is in fact physically and functionally coupled to the β1 integrins in these cells (Levite et al, 2000; Levite 2001). Of note is an interesting difference: in the case of the Kv1.3 channel we previously studied, it is the opening of a Kv1.3 channel that induces T-cell function (i.e., triggers T-cell adhesion) (Levite et al, 2000; Levite 2001), while in the present study, it is the blocking of the Kv1.1 channel that does so (i.e., elevates TNF-α).

Some Human Neurological Diseases Associated With KV1.1 Block: Blocking of the Kv1.1 channel, which in general increases neuronal excitability, is implicated directly in at least three human neurological diseases:

1) PNH associated with Kv1 Abs,

2) Encephalitis associated with Kv1 Abs,

3) Episodic-ataxia type 1 (EA-1) associated with different mutations of the gene that codes for the human Kv1.1 channel (see Vincent et al, 2004; Adelman et al 1995; Browne et al, 1994.

PNH: A Human Neurological Disease Associated with Kv1 Block The deletion of the Kv1.1 channel in mice causes epilepsy, temperature-sensitive shaking and hyperalgesia, features consistent with neuronal hyperexcitability (Smart et al, 1998; Zhou et al, 1998). Clinicians use many terms including neuromyotonia, Issacs' syndrome, myokymia and cramp-fasciculation syndrome, to describe the motor manifestations of generalized PNH (Hart et al, 2002). The idea that PNH is commonly autoimmune-mediated (Newsom-Davis et al, 1993) was recently confirmed by the finding that 35% of 60 PNH patients had serum Kv1 Ab titers above 100 pmol/l (Hart et al, 2002). These Abs are pathogenic and block Kv1 channels, thus inducing nerve hyperexcitability and a spectrum of clinical manifestations in both the peripheral and central nervous system. Here, it has been found that the serum of some Kv1 Ab-positive PNH patients (see Methods) stimulated substantial TNFα secretion by human T-cells, while not elevating IFN-γ (similar to the results found for DTX-K). This finding raises the possibility that Ab-mediated cytokine induction in T-cells may contribute to the pathogenesis of nerve hyperexcitability and the clinical features of PNH and, perhaps, autoimmune encephalitis. The present data also raise the possibility that some of the more non-specific symptoms associated with PNH, such as tiredness and muscle aches, could at least in part be caused by Kv1 Ab-mediated increases in circulating TNF-α. Thus, it is another feature of the present invention to treat 1) PNH associated with Kv1 Abs, 2) encephalitis associated with Kv1 Abs, and 3) episodic-ataxia type 1 (EA-1) by any known means of diminishing the TNF-α titre in the circulation, such as by means of TNFα binding proteins, such as TBP-1 and TBP-2, anti-TNFα antibodies, Kv1.1 channel unblockers, etc.

As T-cell Kv1.1 dysfunction may contribute to human disorders associated with excess TNF α, Kv1.1 in T-cells is a novel therapeutic target. Human TNFα is synthesized as a 233aa, 26 kDa membrane-associated protein with biological activity. The membrane-associated protein is enzymatically cleaved by TNFα converting enzyme (TACE), an adamalysin, to a 157aa, 17 kDa soluble protein that readily homotrimerizes. Binding of the homotrimeric TNF-α (either cell-associated or soluble) to its receptors (TNF-RI and TNF-RII) induces oligomerization of the receptors required for signal transduction in quiescent cells.

Inappropriate or over-production of TNF-α occurs in a wide range of conditions that involve inflammation in their pathogenesis, among them inflammatory bowel disease, rheumatoid arthritis, multiple sclerosis, ankylosing spondylitis and stroke (Baugh et al, 2001). The present findings lead to the likely conclusion that T-cell Kv1.1 block, whether mediated in vivo by Kv1.1 Abs or Kv1.1 blockers, is responsible, at least in part, for the excess TNFα in these disorders, and therefore is a novel therapeutic target.

On the other hand, as TNF-α is crucially needed for many other purposes, passive transfer in humans of autologous DTX-K-treated T-cells that overexpress TNF-α for prolonged periods of time, will be beneficial in very specific circumstances. These may include augmenting T-cell eradication of cancer, boosting T-cell attack of viruses and bacteria (especially in cases of various types of immunodeficiency), and increasing the recruitment of host cells into the liver when needed.

### Example 1

### Materials and Methods

Ion Channel Blockers: See also Table 1. Specified for each ion channel blocker used herein are its full name, its abbreviation and manufacturer in brackets, and its effective concentrations (derived from the respective manufacturer's data sheets and/or the literature). Further detailed information can be found at the International Union of Pharmacology website (see Gutman et al, 2003). The blockers included: 4-Aminopyridine (4-AP, Sigma, St. Louis, Mo.), 10 μM-1 mM; Tetraethylammonium (TEA, Sigma), 100 μM-10 mM; Quinine (Sigma), 1-10 μM; Clotrimazole (CLT, Agis Industries, Bnei Brak, Israel), 10 nM-10 μM; rCharybdotoxin (CTX, Alomone labs, Jerusalem, Israel), 10-100 nM; Kaliotoxin (KTX, Alomone), 1-100 nM; rMargatoxin (MgTX, Alomone), 50 pM-50 nM; Dendrotoxin-K (DTX-K, Alomone), 10-100 nM; Tetrodotoxin (TTX, Alomone), 100 nM-1 μM; NPPB (Tocris Cookson, Avonmouth, UK), 100-200 μM; R-(+)-Bay K8644 (+Bay K, Tocris) 10 nM-1 μM; CNQX (Tocris), 100 nM-50 μM.

TABLE 1
Characteristic Features of the 12 Ion-Channel Blockers used in
the Study

| Ion Channel | | | |
|---|---|---|---|
| Blocker | Abbreviation | Ion Channels Affected | Specific Activity |
| 4-Aminopyridine | 4-AP | K⁺ Channels | blocks K⁺ channels |
| Clotrimazole | CLT | K⁺ Channels | blocks intermediate |
| | | | conductance Ca²⁺ |
| | | | activated k⁺ channels |
| Tetraethylammonium | TEA | K⁺ Channels | blocks Ca²⁺ activated |
| | | | and voltage-gated K⁺ |
| | | | channels |
| rCharybdotoxin | CTX | K⁺ Channels | blocks high/intermediate |
| | | | conductance Ca²⁺ |
| | | | activated and voltage- |
| | | | gated Kv1.3 K⁺ channels |
| Quinine | Quinine | K⁺ Channels | blocks Kv1.3 voltage- |
| | | | gated K⁺ channels |
| rMargatoxan | MgTX | K⁺ Channels | blocks Kv1.3 voltage- |
| | | | gated K⁺ channels |
| Kaliotoxin | KTX | K⁺ Channels | blocks some Ca²⁺ |
| | | | activated and voltage- |
| | | | gated K⁺ channels |
| Dedrotoxin-K | DTX-K | K⁺ Channels | blocks Kv1.1 voltage- |
| | | | gated K⁺ channels |
| Tetrodotoxin | TTX | Na⁺ Channels | blocks excitable Na⁺ |
| | | | channels |
| NPPB | NPPB | Cl⁻ Channels | blocks CT channels |
| (R)-(+)-Bau L 8644 | +Bay K | Ca²⁺ Channels | blocks L-type Ca²⁺ |
| | | | channels |
| CNQX | CNQX | Ionotropic Glutamate | blocks AMPA / Kainate |
| | | Receptors | receptor channels |
| | | (Na⁺/K⁺/Ca²⁺ Channels) | |

Human T-Cells: Normal human T-cells were purified from peripheral blood of healthy donors as described (Levite et al, 2000; Ganor et al, 2003; Levite t al, 1998), and the resulting cell population, containing ≧90% T-cells, was suspended in RPMI medium supplemented with 10% FCS, 1% L-Glutamine and antibiotics (Biological Industries, Bet Haemek, Israel) and maintained at 2×10⁶ cells/ml (37° C./5% CO₂).

Determination of TNF-α, IFN-γ, IL-10 and IL-4 by ELISA: Freshly-purified normal resting human T-cells (2×10⁶/ml) were incubated in 24-well plates (Costar, Corning, N.Y.) and the various channel blockers were added. Importantly, no other stimulating molecule (e.g., phorbol esters, antigens, anti-CD3/CD28 mAbs) was added. Cytokine levels were measured in supernatants after 24 hours for TNF-α and IFN-γ and after 72 hours for IL-10 and IL-4, by quantitative sandwich ELISA (R&D, Minneapolis, Minn.), according to the manufacturer's instructions.

RT-PCR for TNF-α and Kv1.1: Total RNA was prepared according to Tri-Reagent protocol (MRC, Cincinnati, Ohio). First-strand cDNA was synthesized by Reverse Transcription System (Promega, Madison, Wis.). RT-PCR was conducted in a 50 μl reaction mixture containing 50 ng cDNA, 5 μl of X10 Optibuffer (Bioline, London, UK), 3 μl of 50 mM MgCl₂, 2.5 μl of 10 mM dNTP mix (Promega), 4U of Bio-X-act DNA Polymerase (Bioline). For TNF-α/S14 parallel amplification, 2 μl of each TNF-α primer (10 pmol/μl) and 0.25 μl of each S14 primer (10 pmol/μl) were used. For Kv1.1/S14 parallel amplification, 5 μl of each Kv1.1 primer (100 pmol/μl) and 2.5 μl of each S14 primer (10 pmol/μl) were used. The sequences of the primers (5′-3′) and lengths of the products were as follows: TNF-α primer pair—upstream primer CTGAAAGCATGATCCGGGACGTG (SEQ ID NO:1), downstream primer TGACCTTGGTCTGGTAGGAGACG (SEQ ID NO:2), 494 bp; Kv1.1 primer pair—upstream primer GTCACTGTCAGAGGCTAAGTT (SEQ ID NO:3), downstream primer GCATCGACAACACCACGGTC (SEQ ID NO:4), 709 bp; S14 primer pair—upstream primer GTCCATGTCACTGATCTTTCTGGC (SEQ ID NO:5), downstream primer GTTTGATGTGTAGGGCGGTGATAC (SEQ ID NO:6), 166 bp. Conditions for TNF-α PCR were: 94° C. for 1 minute, 63° C. for 40 seconds and 72° C. for 40 seconds (29 cycles). Conditions for Kv1.1 PCR were: 94° C. for 3 minutes (one cycle); 94° C. for 1 minute, 60/57/54/51/48° C. for 2 minutes and 72° C. for 3 minutes (three cycles each); 94° C. for 1 minute, 50° C. for 2 minutes and 72° C. for 3 minutes (25 cycles). Densitometric analysis of the TNF-α/S14 RT-PCR products was performed by Adobe Photoshop 7.0 ME. The relative intensity of the TNF-α bands was normalized to that of the S14 bands.

EMSA for Nuclear NF-κB: Nuclear fractions from freshly-isolated normal human T-cells were collected using Nuclear Extract Kit (Active Motif, Carlsbad, Calif.), according to the manufacturer's instructions. NF-κB levels in nuclear fractions were measured using TransAM NF-κB Family Kit (Active Motif), according to the manufacturer's instructions.

Immunofluorescent Histochemistry for NF-κB: Freshly-isolated normal human T-cells that were pretreated or not with DTX-K (100 nM for 7, 15, 30 and 60 minutes), were pelleted (1200 g, 10 minutes, 40° C.), suspended in PBS at 1×10⁶ cells/ml, centrifuged (1200 g, 10 minutes), fixed on glass slide by cytospin (250 μl/slide, 1500 rpm, 5 minutes; Shandon Elliot, London, UK), permeablized (200 μl/slide of a solution containing 100 ml 95% methanol and 3 ml acetic acid glacial), and incubated in a blocking medium (200 μl/slide) containing 10% normal goat serum, 2% BSA, 1% glycine and 0.5% triton X-100. The cells were then stained with a rabbit anti-human NF-κB p50 polyclonal Ab (80 μl of 1:50 dilution for 30 minutes at room temperature; Santa Cruz Biotechnology, Santa Cruz, Calif.). Following this initial staining, the cells were exposed to a FITC-conjugated goat anti-rabbit IgG (1:50 dilution for 30 minutes at room temperature; Jackson ImmunoResearch, West Grove, Pa.), washed twice in PBS and then exposed to Hoechst 33342 that incorporates to the cell nucleus (1:2000 dilution for 5 minutes at room temperature; Molecular Probes, Eugene, Oreg.). The stained cells were visualized by a fluorescent microscope (Nikon Eclipse E600), and photographs were taken at ×100 magnification using ACT-1 software program.

Immunostaining and Flow Cytometry for Detection of the Kv1.1 Subunit Containing Channels: Freshly-purified normal human T-cells were permeabilized (70% ethanol, 1 hour at −20° C.), and then subjected to immunofluorescence staining, using as the first Ab rabbit anti-Kv1.1 polyclonal Ab (Alomone) at 6 μg/ml/1×10⁶ cells/100 μl for 30 minutes on ice. For isotype control, cells were stained with the same concentration of normal rabbit IgG, prepared as follows: an equal amount of a saturated ammonium sulfate solution was added dropwise to serum obtained from a healthy control white New-Zealand male rabbit; the mixture was stirred for 1 h at 4° C., and centrifuged at 3000 g for 15 minutes at 4° C. The pellet was resuspended in phosphate buffered saline (PBS), and dialyzed three times overnight against the same solution to remove any traces of ammonium sulfate. Estimation of IgG concentration was determined at O.D280 (when 1.45 O.D280=1 mg IgG/ml). The cells were then stained with a FITC-conjugated goat anti-rabbit IgG as a secondary Ab (100 μl of 1:100 dilution; Jackson). Cells that were stained only with the second Ab served as additional negative controls. Fluorescence profiles were recorded in a FACSort.

PNH Patients and Healthy Control Individuals: Serum was collected from 12 PNH patients, all with generalized acquired disease confirmed by electromyography and no clinical or electrophysiological evidence of polyneuropathy. None of the patients had thymus hyperplasia or thymoma on post contrast CT mediastinum. No patient had immunotherapy for at least one year before serum sampling. Serum was also donated by four age, sex and geographically-matched healthy control individuals. Written informed consent for the study was obtained from all patients and the work was approved by the Sefton Research Ethics Committee, Liverpool, UK. Serum Kv1 Ab titers were detected by a standard (125)I-alpha-dendrotoxin immunoprecipitation assay (Hart et al, 1997). This method detects antibodies to Kv1.1, 1.2, and 1.6. Positive titers are set at 100 pM or higher. Titers below 50 pM are considered negative. Seven out of the twelve PNH patients tested herein were positive for Kv1 Abs. The relevant clinical features of the PNH patients are summarized below.

PNH Kv1 Ab-Positive (PNH⁺)

PNH⁺ (1): Female, age 40 y, PNH (onset at 36 y) and Hypothyroidism, Kv1 239 pM.

PNH⁺ (2): Female, age 49 y, PNH only (onset at 47 y), Kv1 335 pM.

PNH⁺ (3): Male, age 52 y, PNH (onset at 40 y) and DM type1, Kv1 235 pM.

PNH⁺ (4): Male, age 52 y, PNH only (onset at 43 y), Kv1 382 pM.

PNH⁺ (5): Male, age 51 y, PNH (onset at 51 y) and mild Psoriasis, Kv1 346 pM.

PNH⁺ (6): Male, age 44 y, PNH only (onset at 43 y), Kv1 165 pM.

PNH⁺ (4): Male, age 23 y, PNH only (onset at 22 y), Kv1 255 pM.

PNH Kv1 Ab-Negative (PNH⁻)

PNH⁻ (7): Female, age, 48 y, PNH only (onset at 41 y).

PNH⁻ (8): Female, age 45 y, PNH (onset at 37 y) and Raynaud's.

PNH⁻ (10): Male, age 53 y, PNH only (onset at 50 y).

PNH⁻ (11): Male, age 49 y, PNH only (onset at 44 y).

PNH⁻ (12): Male, age 41 y, PNH (onset at 15 y) and Eczema.

Testing the Ability of commercial Kv1.1 Ab or Sera of PNH Patients, with or without Kv1 Abs, to Trigger TNF-α Secretion by Normal Human T-Cells: Freshly purified normal human T-cells (2×10⁶/ml) were incubated in 24-well plates and exposed to the rabbit anti-Kv1.1 polyclonal Ab (1:1000 dilution; alomone) for 24 hours. Levels of TNF-α secreted to the culture media were then examined by ELISA as described above.

Alternatively, the T-cells (2×10⁶/ml) were exposed to serum (1:100 dilution) from Kv1 Ab-positive PNH patients, Kv1 Ab-negative patients, or healthy control individuals. Cultures devoid of T-cells served as negative control. Following 24 hours incubation, the levels of TNF-α and IFN-γ in supernatants were examined as described above. TNF-α levels secreted to the culture media were considered elevated if exceeded the mean±2*SD TNF-α level secreted by T-cells exposed to sera of the anti-Kv1.1 Ab-negative PNH patients.

In Vivo Injection of DTX-K-Treated Human T-Cells into SCID Mice: NOD/SCID female mice were injected intraperitoneally (ip) with 200 μl cyclophosphamide (Sigma, 20 mg/ml solution in PBS) 24 hours prior to injection of cells. Freshly-purified normal human T-cells (1×10⁸ cells) were pretreated with DTX-K (100 nM, 24 hours) or PBS, washed, and fluorescently-labeled with 2′,7′-bis-(2-carboxyethyl)-5-(and -6)-carboxyfluorescein acetoxymethyl (BCECF AM, Molecular Probes, Eugene, Oreg.). The human T-cells were then washed, resuspended in PBS, and 200 μl of the labeled cells (equals to 1.5×10⁷ cells) were injected ip to each animal (n=5 for each group). After 24 hours, blood was withdrawn from each animal, and the liver, spleen, kidney, bone marrow and brain were removed. Single cell suspensions were prepared from all organs except the brain. The total number of cells, as well as the number of only the resident SCID cells (non-fluorescent) or only the number of the injected human T-cells (fluorescent) present in each organ was counted by FACSort.

Brain Immunohistochemistry: Brains were removed, immersed in Bouin's fixative solution overnight at 4° C., washed in PBS, and stored in 70% ethanol until paraffin embedding. Serial 7μ thick coronal sections were then cut, deparaffinized, dehydrated and submitted to microwave treatment, using a domestic Samsung microwave oven. This was performed by immersion in 1 mM EDTA buffer (pH=8.0), maximal heating until boiling, additional heating for 10 minutes at 20% efficiency, and a 30 minutes cool period at room temperature in the same buffer.

For immunohistochemistry, sections were preincubated overnight at 4° C. with PBS/20% horse serum (HS) (Vector Laboratories, Burlingame, Calif.). Sections were then incubated for 96 hours at 4° C. with rat anti-human CD3 monoclonal Ab (1:50 in PBS/2% HS; Serotec, Oxford, UK). Following washing with PBS, sections were incubated for 1.5 hours at room temperature with a biotinylated anti-rat IgG (1:100 in PBS/2% HS; Vector), washed again, and then incubated with Cy3-conjugated streptavidin (1:200 in PBS; Jackson). Sections were examined by a fluorescence microscope (Nikon Eclipse E600), and photographs were taken at ×40 magnification by using ACT-1 software program.

Statistical Analysis Statistical significance was analyzed by Student's t-test. For comparing quantitative variables among the different groups of human patients, the non-parametric Kruskal-Wallis test was used; subsequent pairwise comparisons were performed by the nonparametric Mann-Whitney U-test.

### Example 2

### Selective Block of Kv1.1 Subunit Containing Channels in Normal Human T-Cells Triggers Marked and Isolated TNF-α Secretion

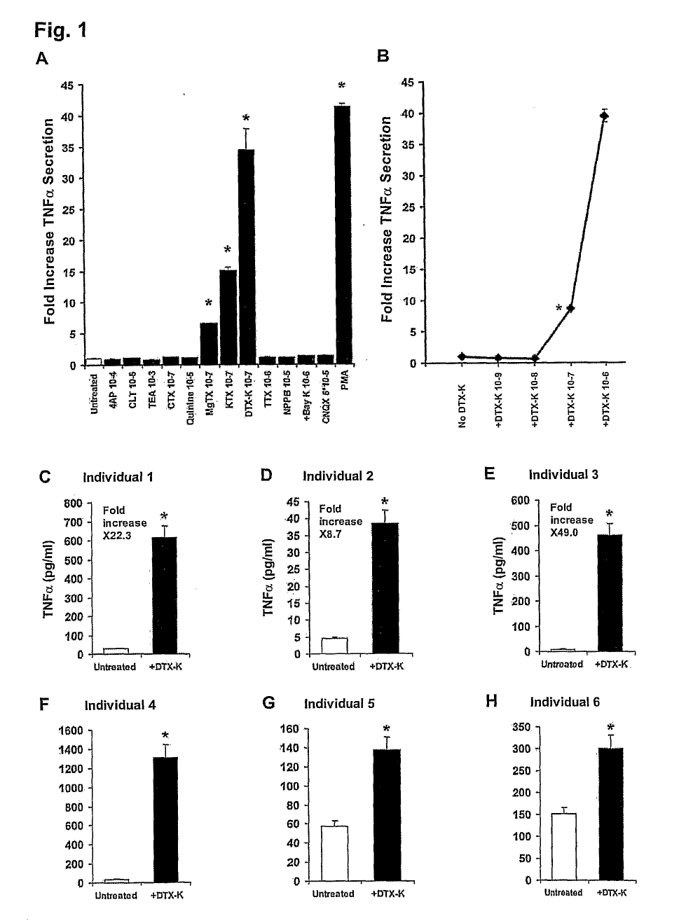

Freshly-purified normal human T-cells (“resting” normal human peripheral T-cells) were exposed to 12 different ion channel blockers (see Table 1) in the complete absence of any other stimulating molecules. These 12 blockers were selected on the basis of their reported ability to block most types of K⁺, Na⁺, Ca²⁺ and Cl⁻ channels expressed in T-cells (Deutsch et al, 1986; Lewis et al, 1995; Lewis et al, 1988; Rader et al, 1996; Freedman et al, 1995; Koo et al, 1997; Lin et al, 1993; Ishida et al, 1993; Jensen et al, 1999; Kotturi et al, 2003; Phipps et al, 1996; Verheugen et al, 1997; Levite et al, 2000; Chandy et al, 1984). The levels of TNFα secreted into the culture media were tested 24 hours later by ELISA. For positive control, the cells were exposed to the potent phorbol ester PMA, know to activate T-cells in a non-specific manner. The results are shown in FIG. 1A and represent fold increase ±SD TNF-α secretion tested in 2-3 independent experiments. Each ion channel blocker was examined in at least two independent experiments, and at several concentrations covering its effective range (see Materials and Methods). The numbers in each figure next to each blocker represent the highest molar concentration used. Statistical analysis: *P=0.0001, 0.0005, 0.0011 and 0.0005 vs. untreated for MgTX, KTX, DTX-K and PMA, respectively (Student's t-test).

FIG. 1A reveals that block of the Kv1.1 subunit containing voltage-gated K⁺ channels by Dendrotoxin-K (DTX-K), a highly selective Kv1.1 blocker (Harvey et al, 2001) (and used as such in numerous studies thus far, some of which cited in (Harvey et al, 2001)), triggered by itself a marked TNF-α secretion. DTX-K, used at an optimal recommended effective concentration (i.e., 100 nM, as commonly used in many other studies), induced 33.4 fold increase in TNFα secretion by the studied human T-cells, a comparable magnitude of elevation obtained by the potent non-specific activator phorbol ester (PMA, 41.3 fold increase), used herein for positive control. Significant augmentation in TNFα secretion was also induced by Kaliotoxin (KTX) and rMargatoxin (MgTX) (15.1 and 6.5 fold increase, respectively, FIG. 1A), which are blockers of voltage gated K⁺ channels, affecting predominantly the Kv1.3 channel, but which may also affect the Kv1.1 channels. In comparison, DTX-K has much higher affinity and selectivity to Kv1.1 than KTX and MgTX (e.g., the Ki for Kv1.1 are 16 pM for DTX-K, and markedly higher: 20 nM for KTX (Gutman et al, 2003)).

In contrast to DTX-K, KTX and MgTX, no significant TNF-α up-regulation was observed following exposure of the normal human T-cells to several effective concentrations of nine other ion channel blockers (Table 1), among them blockers of additional K⁺ channels (which are much less selective for Kv1.1): 4-Aminopyridine (4-AP), Clotrimazole (CLT), Tetraethylammonium (TEA), rCharybdotoxin (CTX) and Quinine; a Na⁺ channel blocker: Tetrodotoxin (TTX); a Cl⁻ channel blocker: NPPB; a Ca²⁺ channel blocker: R-(+)-Bay K8644 (+Bay K); and the blocker of the ion channel (ionotropic) glutamate/AMPA receptor (which was recently identified in high levels in human T cells (Ganor et al, 2003)): CNQX.

The triggering effect of DTX-K was dose dependent, as shown in FIG. 1B. Shown is the fold increase ±SD TNFα secretion following 24 hour exposure to 10⁻⁹ M-10⁻⁶ M DTX-K. One representative experiment of three performed is shown. *P=0.0001 vs. untreated (Student's t-test). The increase in TNFα secretion was 8.7 fold for 100 nM DTX-K and 39.5 fold for 1 μM DTX-K (of note, the results presented in FIGS. 1A and 1B are of T-cells purified from two different human individuals, see below).

In subsequent experiments, we tested freshly isolated T-cells from ten additional human individuals, and found that the effect of DTX-K was reproducible. Shown in FIGS. 1C-H are the actual mean concentrations of TNF-α in pg/ml±SD (rather than fold increase drawn in a and b) secreted to the culture media following exposure (24 hours, 100 nM) to DTX-K of the T-cells from these six human individuals. Statistical analysis: *P=0.0054, 0.0052, 0.0063 and 0.0197 vs. untreated for the individuals 1, 3, 5-6, respectively (Student's t-test). TNF-α secreted by T-cells of individuals 4 and 2, shown herein in pg/ml, was also shown above in fold increase (FIGS. 1A and B, respectively). FIGS. 1C-H show that despite the different background levels of T-cell derived TNF-α, DTX-K (24 hours, 100 nM) triggered marked TNF-α secretion by T-cells of all six individuals, resulting in fold increases of 22.3, 8.7, 49.0, 34.4, 2.4 and 2.0, respectively. The highest DTX-K-induced TNF-α elevation was from 38 pg/ml to 1308 pg/ml, reflecting a 34.4 fold increase (FIG. 1F, individual 4). Of note, DTX-K had no significant effect on cell survival or proliferation of these human T-cells (data not shown).

### Example 3

### Blocking the Kv1.1 Subunit Containing Channels by DTX-K Induces an Exclusive Secretion of TNF-α, while “Classical” TCR-Activation Induces a Robust Yet Non-Exclusive Secretion of TNF-α, IFN-γ, IL-10 and IL-4

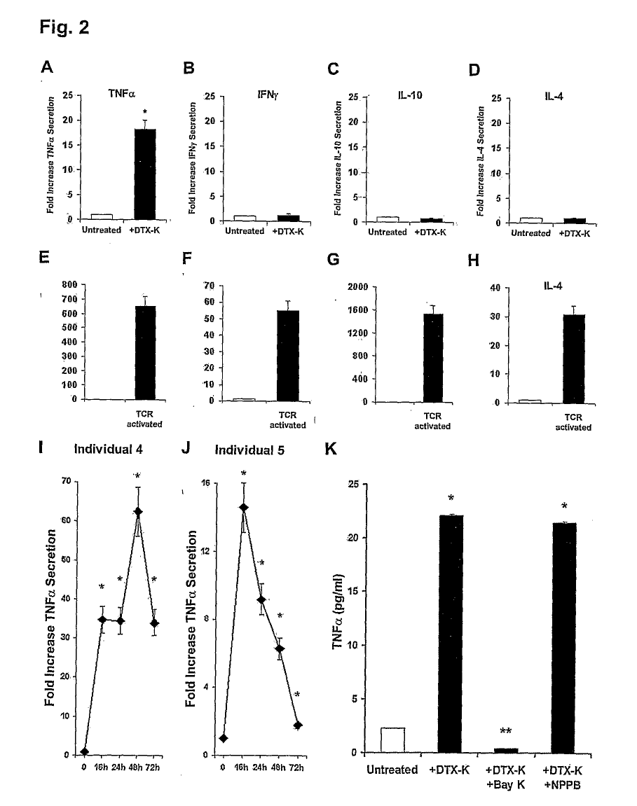

In FIGS. 2A-H, normal human T-cells were either exposed to 100 nM DTX-K (FIGS. 2A-D), or to “classical” TCR-activation using anti-CD3 and anti-CD28 mAbs (FIGS. 2E-H). The levels of TNF-α, IFN-γ, IL-10 and IL-4 secreted to the culture media within the next 24-72 hours were then examined by ELISA. Shown are the fold increases ±SD of TNFα (FIGS. 2A and E, 24 hr), IFN-γ (FIGS. 2B and 2F, 24 hours), IL-10 (FIGS. 2C and 2G, 72 hours) and IL-4 (FIG. 2D and sH, 72 hours) secretion from one representative experiment, of at least three independent experiments performed. Statistical analysis: *P=0.0056 (A), 0.0050 (E), 0.0051 (F), 0.0050 (G) and 0.0053 (H) vs. untreated (Student's t-test).

Interestingly, it was found that blocking Kv1.1 by DTX-K elevated only TNFα, while not affecting the levels of IFN-γ, IL-4 and IL-10 (FIGS. 2A-D, expressing the results in fold increase secretion of all four cytokines). In sharp contrast, TCR-activation, achieved by anti-CD3 and anti-CD28 Abs, induced a robust yet non-selective secretion of all four Th cytokines (FIG. 2E-2H). As expected, the magnitude of TNF-α secretion after TCR-activation was much higher than that obtained after DTX-K treatment (FIG. 2E vis-a vis FIG. 2A). Blocking the Kv1.1 subunit containing channels in T-cells by DTX-K either during or after TCR-activation did not cause additional elevation in TNFα levels, beyond that achieved by the TCR-activation alone (data not shown).

### Example 4

### Blocking Kv1.1 Subunit Containing Channels in T-Cells Induces a Prolonged TNF-α Elevation

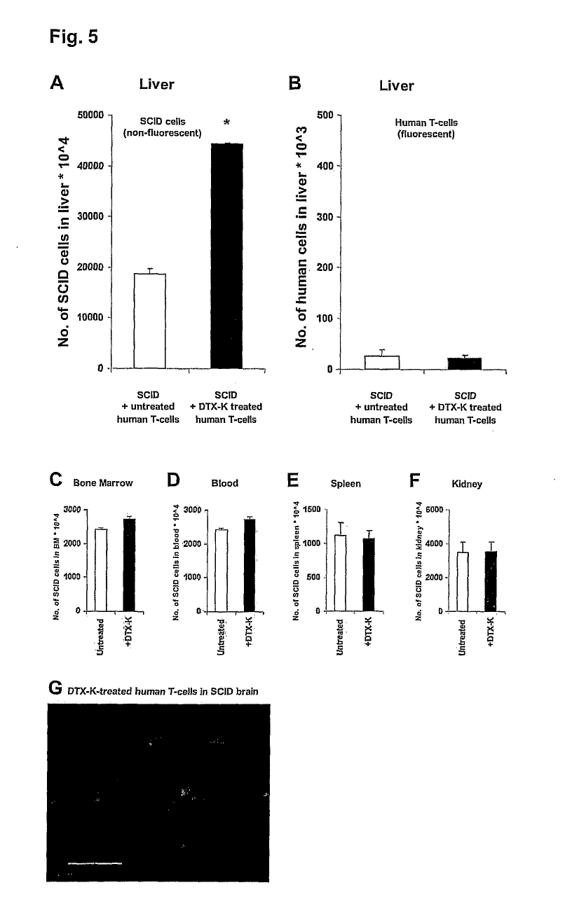

TNF-α secretion by T-cells peaks typically 24 hr after TCR-activation and declines gradually afterwards. Accordingly, the TNF-α secretion induced by blocking the Kv1.1 subunit containing channels in T-cells was investigated to see if they also peak around this time, and whether the secretion is transient or rather prolonged. In FIGS. 2I and 2J, normal human T-cells from individuals 4 (FIG. 2I) and 5 (FIG. 2J), the same two individuals shown already in FIG. 1F, 2G) were exposed to 100 nM DTX-K, and the culture media were examined for TNF-α by ELISA at different time points thereafter. Fold increase ±SD TNF-α secretion for each time point is shown for each individual. *P=0.0053, 0.0052, 0.0050 and 0.0053 vs. untreated for 16, 24, 48 and 72 hours of individual 4, respectively; *P=0.0057, 0.0063, 0.0072 and 0.0316 vs. untreated for 16, 24, 48 and 72 hours of individual 5, respectively (Student's t-test).

This experiment reveals that there is somewhat of a difference in the kinetics of DTX-K-induced TNFα secretion (FIGS. 2I, 2J), as compared to their background and DTX-K-induced T-cell derived TNFα levels (FIGS. 1F, 2G). Despite these differences, they had two features in common: 1) significantly elevated TNFα secretion was evident already after 16 hours; 2) TNF-α secretion remained significantly high even after 72 hours from the onset of DTX-K treatment.

### Example 5

### DTX-K-Induced TNF-α Secretion Requires Ca²⁺ Influx Across the T-Cell Membrane

Ca²⁺ is known to be involved in TNF-α transcription (Lobo et al, 1999). On this basis, it was investigated whether the marked TNF-α secretion induced by blocking Kv1.1 subunit containing channels in T-cells requires “uninterrupted” Ca²⁺ influxes. A potent and very selective blocker of L-type Ca²⁺ channels expressed in T-cells was used: (R)-(+)-Bay K 8644, and added to normal human T-cells 5 minutes before the addition of DTXK (100 nM). It was found that the selective Ca²⁺ channel blocker prevented completely the ability of DTX-K to induce TNF-α secretion, while the addition of the control Cl− channel blocker, NPPB (Table 1), had no such interrupting effect (FIG. 2K). In this experiment, TNFα secretion in response to Kv1.1 block (100 nM DTX-K, 24 hours) is completely prevented if the T-cells are pretreated with 100 μM +Bay-K, the highly selective blocker of Ca²⁺ channels expressed in T-cells (added 5 min before DTX-K), but not if pretreated with 100 μM of the non-relevant Cl⁻ channel blocker NPPB. Results represent mean (pg/ml)±SD TNF-α secretion from one experiment of three performed. Statistical analysis: *P<0.0001 vs. untreated, **P<0.0001 vs. DTX-K alone (Student's t-test).

### Example 6

### Blocking Kv1.1 Subunit Containing Channels in T-Cells Elevates also TNF-α mRNA Levels

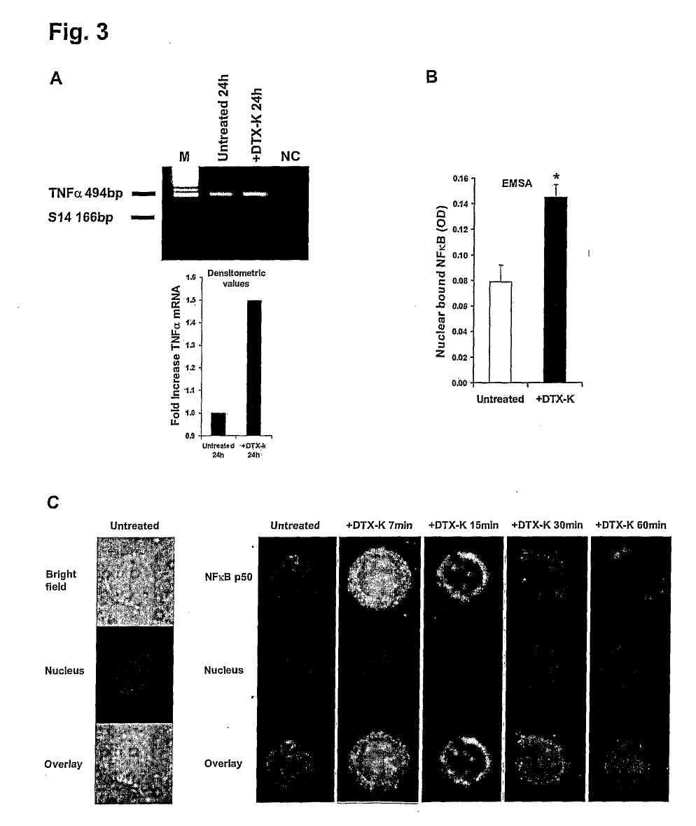

The question of whether selective block of Kv1.1 subunit containing channels in T-cells elevates not only the levels of the TNF-α protein, but also TNF-α mRNA was studied next. For studying this question, DTX-K was added to normal human T-cells for 24 hours, RNA was prepared and transcribed into cDNA, and then specific TNF-α RT-PCR was performed, using TNFa-specific primers. For control of the reverse transcription efficiency, an additional RT-PCR reaction using an internal standard: the ribosomal S14 protein, was performed in parallel (i.e., in the same PCR tube). More specifically, normal peripheral human T-cells were exposed to 100 nM DTX-K for 24 hr. cDNA was then prepared and amplified by two RT-PCR reactions performed in parallel in the same PCR tube, using primers specific for TNFα and for the internal control, i.e., the ribosomal S14 protein. The results are shown in FIG. 3A. Upper bands: TNF-α (expected product size: 494 bp); lower bands: S14 (expected product size: 166 bp). Bars at the bottom represent fold increase TNF-α mRNA calculated following densitometric analysis and normalization of the corresponding RT-PCR bands of TNF-α/S14. NC=negative control (no cDNA).

FIG. 3A reveals that indeed, blocking the Kv1.1 subunit containing channels by DTX-K augmented TNFα mRNA levels.

### Example 7

### Blocking Kv1.1 Subunit Containing Channels Leads to an Activation of NF-κB in Normal Human T-Cells

TNF-α (like other proinflammatory cytokines such as IL-1) not only activates NF-κB, but is also induced by NF-κB (Siebenlist et al, 1990). In case of the TNF-α gene, the observed importance of NF-κB for the expression of the TNFα gene may depend on the cell type and the segment of the enhancer/promoter analyzed (Siebenlist et al, 1990). On these grounds, we investigated whether block of Kv1.1 channels in normal human T-cells (once again, in the complete absence of any other stimuli) can affect NF-κB. This was studied by two technologies: 1) preparing nuclear extracts from untreated and DTX-K treated normal human T-cells and measuring the amount of NF-κB bound to nuclear NF-κB-binding sequences by commercially available Electrophoresis Mobility Shift Assay (EMSA); 2) visualizing the putative translocation of NF-κB into the nucleus by immunohistochemistry staining, using anti-human NF-κB p50 specific Ab, FITC-conjugated anti-human IgG, and Hoechst 33342 which stains specifically the cell nucleus.

Nuclear extracts were that was added for several time periods, of which only 7 min is shown. The results, expressed as OD±SD, reflect the amount of NF-κB bound to nuclear NF-κB-binding sequences, as determined by commercially available EMSA. One of two independent experiments is shown in FIG. 3B. Statistical analysis: *P=0.0092 (Student's t-test).

It can be seen from FIG. 3B, in which the OD values represent the amount of NF-κB bound to nuclear NF-κB-binding sequences, that 7 minutes after addition of DTX-K, the level of NF-κB present in the nuclear extracts was significantly augmented. As expected, this elevation was transient, and no longer detected at several later time points tested (data not shown). The findings detected by EMSA were in line with the double immunofluorescence photographs shown in FIG. 3C. In the experiment the results of which are shown in FIG. 3C, normal human T-cells were either left untreated or treated with 100 nM DTX-K for 7, 15, 30 and 60 minutes (see DTX-K treatment time above each respective column). Immunohistochemical staining was performed using either anti-human NF-κB p50 Ab followed by FITC-conjugated anti-human IgG, or Hoechst 33342 which incorporates to the cell nucleus. Representative photographs at ×100 magnification are shown for NF-κB p50 green fluorescence (upper panel), nuclei blue fluorescence (middle panel), and overlay of both (lower panel). The overall sizes of the studied T-cells (bright field) and nucleus (Hoechst) are shown in the left column. Thus, FIG. 3C reveals that 7 minutes after DTX-K addition (third column from left), the cell nucleus (Hoechst blue fluorescence) is clearly positive for NF-κB p50 (green fluorescence), which was not the case in the untreated cells (FIG. 3C, second column from left), and also in three later time points tested: 15, 30 and 60 minutes (FIG. 3C, three right columns, respectively). Of note, 7 minutes after DTX-K treatment, also the cytoplasm (Hoechst negative) was positively stained for NF-κB p50 (FIG. 3C, third column from left), while for some still unclear reason, no cytoplasmatic NF-κB p50 was observed in the untreated cells. The only logical, yet still speculative, explanation that comes to mind now is that the anti-p50 Ab used herein is somewhat sensitive to conformation, and if so, after the activation and release of the NF-κB inhibitor IkB (Siebenlist et al, 1990), the cytoplasmatic p50 may be detected better by this Ab.

### Example 8

### Normal Human T-Cells Express the Voltage-Gated Kv1.1 Protein and mRNA

Previous studies have shown that functional (i.e., electrophysiologically active) Kv1.1 subunit containing channels are expressed primarily in the brain, heart, retina, skeletal muscle and islets (Beckh et al, 1990; Klumpp et al, 1991; Matsubara et al, 1991; Roberds et al, 1991; Tsaur et al, 1992). In contrast, as to Kv1.1 currents in T-cells, the picture arising from previous studies is not as clear. Some studies, testing by patch-clamp techniques, whether human T-cells express Kv1.1 characteristic, failed to identify such currents (e.g., (Cahalan et al, 1985)). Yet, in contrast, Freedman et al showed Kv1.1 currents/expression by murine CD4-CD8-thymocytes (Freedman et al, 1995) and Liu et al reported that currents expressed in naive mice CD4⁺ lymphocytes are consistent with Kv1.1, Kv1.2, Kv1.3, and Kv1.6, based on the use of several methodologies (Liu et al, 2002). Herein, studying a completely novel facet of the Kv1.1 protein, it was desired to confirm the expression of Kv1.1 protein and mRNA in normal peripheral human T-cells (without testing again for Kv1.1 currents, which is out of the scope of the present study). The present methodology included Kv1.1 specific RT-PCR, using Kv1.1 specific primers, as well as flow cytometry (a methodology not used thus far for this purpose), using Kv1.1 specific Abs.

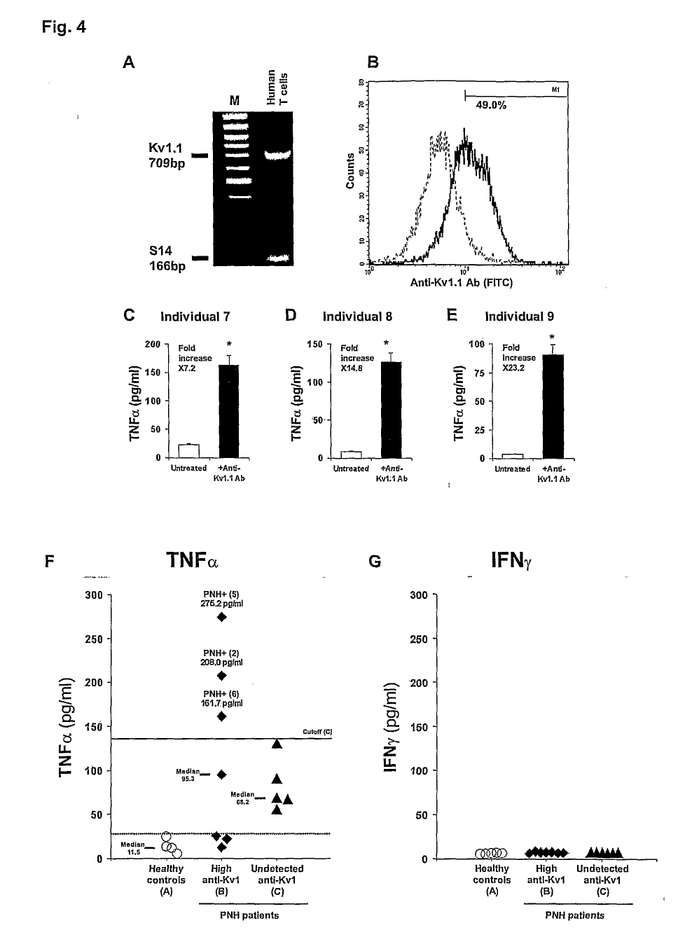

FIG. 4A shows the results of an experiment in which cDNA was prepared from normal peripheral human T-cells, and Kv1.1 specific RT-PCR was performed, using Kv1.1-specific primers (upper band; expected product size: 709 bp). Control RT-PCR was performed in parallel in the same PCR tube using ribosomal S14 primers (lower band; expected product size: 166 bp). FIG. 4A shows that Kv1.1 specific mRNA was indeed amplified from the cDNA of the normal human T-cells. In addition, Kv1.1 specific immunofluorescent staining was performed. T-cells were stained either with a rabbit anti-Kv1.1 polyclonal Ab (FIG. 4B, solid bold line), or, as isotype control non-specific staining, with normal rabbit IgG (FIG. 4B, dashed line), and then with FITC-conjugated anti-rabbit IgG. Kv1.1 expression was evaluated by FACSort. One of five independent experiments (using T-cells of different individuals) is shown, and herein 49.0% (M1) of the human T-cells cells were Kv1.1-positive. In the other four experiments performed, the percentage of Kv1.1-positive T-cells varied between 40-80%. Thus, FIG. 4B shows, by flow cytometry analysis, that the Kv1.1 protein is clearly expressed on the cell surface of a substantial proportion of normal peripheral human T-cells.

### Example 9

### Commercial Anti-Kv1.1 Channel Ab Triggers Marked TNF-α Secretion, Thereby Mimicking the Effects of the Selective Kv1.1 Blocker

In this experiment we investigated whether anti-Kv1.1 specific Abs act like the Kv1.1 channel blocker, DTX-K, and elevate TNFα secretion. Normal human T-cells from three additional individuals (individuals 7-9, respectively) were incubated with a commercial rabbit anti-Kv1.1 polyclonal Ab (1:1000 dilution, 24 hours) and the levels of TNFα secreted into the culture medium were tested by ELISA. The results are shown in FIGS. 4C, 4D and 4E, present the mean (pg/ml)±SD TNFα secretion by T-cells of each individual. *P=0.0003, 0.0057 and 0.0021 vs. untreated for individuals 7-9, respectively (Student's t-test).

FIGS. 4C-4E show that the commercial anti-Kv1.1 Ab, incubated for 24 hours with normal human T-cells derived from three different individuals, induced 7.2, 14.8 and 23.2 fold increases respectively in the amounts of TNFα secreted by these cells. Of note, the anti-Kv1.1 Ab used herein is directed against an intracellular epitope of the Kv1.1 channel. It is speculated that this Ab induced TNF-α secretion following its penetration into the T-cells, based on previous reports, which clearly demonstrated the ability of Abs to penetrate into living cells (Alarcon-Segovia et al, 1996; Ruiz-Arguelles et al, 2003). In fact, in various autoimmune diseases, the pathological Abs are directed against intracellular epitopes (e.g., anti-DNA and anti-ribonucleoprotein Abs in systemic lupus erythematosus) (Alarcon-Segovia et al, 1996; Ruiz-Arguelles et al, 2003), and exert their detrimental effects only after penetration into the cells.

### Example 10

### Kv1 Ab-Positive Peripheral Nerve Hyperexcitability (PNH) Serum Drives Marked Secretion of TNF-α by Normal Human T-Cells

This experiment investigates whether the pathogenic Kv1 Abs present in the serum of PNH patients (i.e., Kv1.1, Kv1.2 and Kv1.6 Abs) (Hart et al, 2002) reproduce the actions of commercial Kv1.1 Abs and DTX-K, and also increase TNF-α secretion by human T-cells. The common presenting clinical features of PNH are muscle twitching, cramps, stiffness and weakness, and can be accompanied by hyperhidrosis, pseudomyotonia, pseudotetany, and sensory symptoms (Newsom-Davis et al, 1993). In addition, PNH patients can have CNS features (Vincent et al, 2004). Most cases of PNH are autoimmune and caused by circulating Kv1 Abs (Hart et al, 002), which are a mixture of Kv1.1, 1.2, and 1.6 Abs.

To test this hypothesis, normal peripheral human T-cells were exposed to sera (1:100 dilution) of either healthy control individuals (group A), Kv1 Ab-positive PHN patients (group B), or Kv1 Ab-negative PHN patients (group C). All groups were age, sex and geographically matched (see Methods). The levels of TNF-α (FIG. 4F) or IFN-γ (FIG. 4G) secreted into the medium in the following 24 hours were tested by ELISA. Results represent the mean of three independent experiments performed. In FIG. 4F, the horizontal bars and respective values denote the medians. The dashed line, serving herein as the lower cutoff (cutoff A), represents the mean+2*SD of TNF-α secreted by T-cells that were exposed to the sera of the control group; the solid line, serving herein as the upper cutoff (cutoff C), represents the mean+2*SD of TNF-α secreted by T-cells exposed to the Kv1 Ab-negative PNH sera. The respective numbers shown in the graph denote the actual mean amounts of TNF-α (pg/ml) secreted. Note that sera of three Kv1-positive PNH patients (namely, PNH⁺5, 2 and 6) induced elevated secretion of TNFα (but not IFN-γ), clearly above cutoff C. Statistical analysis: KW statistic=8.0003, P=0.0183, Kruskal-Wallis test. Pairwise comparisons were performed with Mann-Whitney U-test: P=0.0177 groups B vs. A, P=0.0087 groups C vs. A, P=0.6282 groups B vs. C.

FIG. 4F shows that the serum of some Kv1 Ab-positive PNH patients induced marked TNFα secretion, significantly higher than that induced by healthy control individuals, or by serum from PNH patients without detectable Kv1 Abs. Moreover, the TNF-α triggering effect correlated positively with Ab titer (see Materials and Methods). Negative control experimental cultures, which contained the same volume and concentration of serum from PNH patients, but without T-cells, had no effect on TNFα levels. Importantly, Kv1 Ab-positive sera of PNH patients induced elevation of TNFα but not of IFN-γ (FIG. 4G), alike observed for DTX-K (FIG. 2), confirming the selectivity of the effect. Altogether, this set of experiments show that the sera of some PNH patients, containing raised Kv1 Ab titers, induced marked TNFα secretion by normal human T-cells, and as such mimicked the effects of the commercial anti-Kv1.1 Abs and the selective Kv1.1 blocker DTX-K.

### Example 11

### Injection into SCID Mice of Normal Human T-Cells Secreting Elevated TNF-βL Levels Due to Prior Kv1.1 Block Induces Selective Recruitment of Host Cells into the Liver

To test whether the TNF-α secreted by human T-cells in response to Kv1.1 selective block is functional in vivo, we recalled a recent study showing that following administration of TNF-α into the brain, there is a series of events, among them a rapid up to two fold increase in the number of host ED1-positive cells (i.e., resident and activated Kuppffer cells/recruited blood monocytes), which accumulated specifically in the liver (Campbell et al, 2005). We investigated whether the elevated TNFα secreted by DTX-K-treated human T-cells also induces such an effect. For testing this, freshly-purified normal human T-cells were exposed to either PBS or to 100 nM DTX-K for 24 hours, washed, fluorescently-labeled and then, after confirming that the latter cells secrete elevated TN-Fα (data not shown), injected into SCID mice (i.e., devoid of their own lymphocytes) (n=5 for each group). The amount of the total number of cells, as well as only of the resident SCID cells (non-fluorescent) and only of the injected human T-cells (fluorescent) present in single cell suspensions of liver, bone marrow, spleen and kidney, as well as in blood, were counted 24 hours later by FACSort. In regards to the liver, the results represent the mean±SEM number of non-fluorescent SCID host cells (FIG. 5A) and the mean±SEM of the fluorescently-labeled human T-cells (FIG. 5B). In regards to the other organs, only the number of non-fluorescent SCID host cells is shown: bone marrow (FIG. 5C), blood (FIG. 5D), spleen (FIG. 5E) and kidney (FIG. 5F), *P<0.0001 vs. untreated (Student's t-test). FIG. 5G shows immunohistochemistry of brain slices from SCID mice injected with human T-cells preincubated ex vivo with DTX-K, for the detection of the infused human T-cells. A representative photograph at ×40 magnification is presented, showing that 24 hr following injection of DTX-K-treated human T-cells into SCID mice, human CD3-positive cells are detected within the cortex.

FIG. 5A shows that the infusion of TNF-α-secreting DTX-K-treated human T-cells doubled the number of cells in the liver. As shown in the relevant study we relied on (Campbell et al, 2005), this effect was restricted to the liver, as the number of cells in the bone marrow, blood, spleen and kidney did not change significantly (FIGS. 5C-5F, respectively). Moreover, as anticipated, the increased number of cells in the liver resulted from recruitment of mouse (SCID) host cells (FIG. 5A) and not from the passively-transferred human T-cells entering this organ (FIG. 5B). The exact identity of the recruited mouse cells was not further studied due to technical limitations. Finally, immunohistochemistry of brain slices revealed the presence of the injected DTX-treated human T-cells within the cortex of the injected SCID mice (FIG. 5G). This indicates that in principal, the in vivo recruitment of host cells into the liver could be mediated by TNF-α operating within the brain (i.e., TNF-α delivered to the brain by the DTX-treated human T-cells), resembling the recently reported recruitment of host cells into the liver following injection of TNFα into the brain (Campbell et al, 2005). In fact, based on the ability of TNFα to disrupt the BBB (Mayhan 2002), it was of no surprise that DTX-K-treated cells managed to enter the brain. Statistically valid quantitative comparison between the number of untreated and DTX-treated human cells that reached the brains was not possible. Of final note, it was of no surprise to us that the mouse SCID cells recruited to the liver could respond to the human TNF-α (secreted by the DTX-K-treated human T-cells), as previous studies showed that the murine TNF-α receptor type 1 has similar affinity for both recombinant murine and human TNF-α (Lewis et al, 1991). Moreover, transgenic mice expressing human TNF-α develop many features that are characteristic of human patients with rheumatoid arthritis (Keffer et al, 1991; Li et al, 2003), indicating that cells of mouse origin can respond to human TNF-α.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### REFERENCES

Adelman et al. 1995. Episodic ataxia results from voltage-dependent potassium channels with altered functions. Neuron. 15:1449-1454.
Alarcon-Segovia et al. 1996. Broken dogma: penetration of autoantibodies into living cells. Immunol Today 17:163-164.
Arnett et al. 2001. TNF alpha promotes proliferation of oligodendrocyte progenitors and remyelination. Nat Neurosci 4:1116-1122.
Baugh et al. 2001. Mechanisms for modulating TNF alpha in immune and inflammatory disease. Curr Opin Drug Discov Devel 4:635-650.
Beckh et al. 1990. Members of the RCK potassium channel family are differentially expressed in the rat nervous system. Embo J 9:777-782.
Beeton et al. 2001. Selective blocking of voltage-gated K⁺ channels improves experimental autoimmune encephalomyelitis and inhibits T cell activation. J Immunol 166:936-944.
Bortner et al. 1999. Caspase independent/dependent regulation of K(+), cell shrinkage, and mitochondrial membrane potential during lymphocyte apoptosis. J Biol Chem 274:21953-21962.
Browne et al. 1994. Episodic ataxia/myokymia syndrome is associated with point mutations in the human potassium channel gene, KCNA1. Nat. Genet. 8:136-140
Campbell et al. 2005. Central nervous system injury triggers hepatic CC and CXC chemokine expression that is associated with leukocyte mobilization and recruitment to both the central nervous system and the liver. Am J Pathol 166:1487-1497.
Cahalan et al. 1985. A voltage-gated potassium channel in human T lymphocytes. J Physiol 358:197-237.
Chandy et al. 1984. Voltage-gated potassium channels are required for human T lymphocyte activation. J Exp Med 160:369-385
DeCoursey et al. 1984. Voltage-gated K⁺ channels in human T lymphocytes: a role in mitogenesis? Nature 307:465-468
Deutsch et al. 1986. Voltage-gated potassium conductance in human T lymphocytes stimulated with phorbol ester. J Physiol Lond 372:405-423.
Dhawan et al. 2003. BTK-2 a new inhibitor of the Kv1.1 potassium channel purified from Indian scorpion Buthus tamulus., FEBS Lett., 539:7-13.
Dinarello et al. 2002. Proinflammatory and antiinflammatory cytokines in rheumatoid arthritis. Amgen Inc., Thousand Oaks, Calif.
Finsen et al. 2002. TNFalpha: kill or cure for demyelinating disease? Mol Psychiatry 7:820-821.
Freedman et al. 1995. Identification of Kv1.1 expression by murine CD4-CD8-thymocytes. A role for voltage-dependent K⁺ channels in murine thymocyte development. J Biol Chem 270:22406-22411.
Freedman et al. 1992. Evidence for voltage modulation of IL-2 production in mitogen-stimulated human peripheral blood lymphocytes. J Immunol 149:3784-3794.
Ganor et al. 2003. Human T cells express a functional ionotropic glutamate receptor GluR3, and glutamate by itself triggers integrin-mediated adhesion to laminin and fibronectin and chemotactic migration. J Immunol 170:4362-4372.
Grinstein et al. 1990. Calcium-independent cell volume regulation in human lymphocytes. Inhibition by charybdotoxin. J Gen Physiol 95:97-120.
Grissmer, S., et al. 1994. Pharmacological characterization of five cloned voltage-gated K⁺ channels, types Kv1.1, 1.2, 1.3, 1.5, and 3.1, stably expressed in mammalian cell lines. Mol Pharmacol 45:1227-1234.
Gutman et al. 2003. International Union of Pharmacology. XLI. Compendium of voltage-gated ion channels: potassium channels. Pharmacol Rev 55:583-586.
Hart et al. 2002. Phenotypic variants of autoimmune peripheral nerve hyperexcitability. Brain 125:1887-1895.
Hart et al. 1997. Autoantibodies detected to expressed K⁺ channels are implicated in neuromyotonia. Ann Neurol 41:238-246.
Harvey, A. L. 2001. Twenty years of dendrotoxins. Toxicon 39:15-26.
Ishida et al. 1993. Lack of voltage sensitive potassium channels and generation of membrane potential by sodium potassium ATPase in murine T lymphocytes. J Immunol 151:610-620
Jensen et al. 1999. Inhibition of T cell proliferation by selective block of Ca(2+)-activated K(+) channels. Proc Natl Acad Sci USA 96:10917-10921.
Kauferstein et al. 2003. A novel conotoxin inhibiting vertebrate voltage-sensitive potassium channels, Toxicon 42:43-52.
Keffer et al. 1991. Transgenic mice expressing human tumour necrosis factor: a predictive genetic model of arthritis. Embo J 10:4025-4031.
Klumpp et al. 1991. The potassium channel MBK1 (Kv1.1) is expressed in the mouse retina. Cell Mol Neurobiol 11:611-622.
Koo et al. 1997. Blockade of the voltage-gated potassium channel Kv1.3 inhibits immune responses in vivo. J Immunol 158:5120-5128
Kotturi et al. 2003. Identification and functional characterization of voltage-dependent calcium channels in T lymphocytes. J Biol Chem 278:46949-46960.
Leonard et al. 1992. Selective blockers of voltage-gated K⁺ channels depolarize human T lymphocytes: mechanism of the antiproliferative effect of charybdotoxin. Proc Natl Acad Sci USA 89:10094-10098
Levite M. 2001. Nervous immunity: neurotransmitters, extracellular K⁺ and T-cell function. Trends Immunol 22:2-5.
Levite et al. 2000. Extracellular K(+) and opening of voltage-gated potassium channels activate T cell integrin function: physical and functional association between Kv1.3 channels and beta1 integrins. J Exp Med 191:1167-1176.
Levite et al. 1998. Neuropeptides, via specific receptors, regulate T cell adhesion to fibronectin. J Immunol 160:993-1000.
Lewis et al. 1995. Potassium and calcium channels in lymphocytes. Annu Rev Immunol 13:623-653.
Lewis et al. 1991. Cloning and expression of cDNAs for two distinct murine tumor necrosis factor receptors demonstrate one receptor is species specific. Proc Natl Acad Sci USA 88:2830-2834.
Lewis et al. 1988. Subset-specific expression of potassium channels in developing murine T lymphocytes. Science 239:771-775
Li et al. 2003. The TNF-alpha transgenic mouse model of inflammatory arthritis. Springer Semin Immunopathol 25:19-33.
Lin et al. 1993. Voltage-gated potassium channels regulate calcium-dependent pathways involved in human T lymphocyte activation. J Exp Med 177:637-645
Liu et al. 2002. Modulation of Kv channel expression and function by TCR and costimulatory signals during peripheral CD4(+) lymphocyte differentiation. J Exp Med 196:897-909.
Lobo et al. 1999. Calcium-dependent activation of TNF family gene expression by Ca²⁺/calmodulin kinase type IV/Gr and calcineurin. J Immunol 162:2057-2063.
Maeno et al. 2000. Normotonic cell shrinkage because of disordered volume regulation is an early prerequisite to apoptosis. Proc Natl Acad Sci USA 97:9487-9492.
Mathie et al. 1998. Voltage-activated potassium channels in mammalian neurons and their block by novel pharmacological agents. Gen Pharmacol 30:13-24.
Matsubara et al. 1991. Pretranslational mechanisms determine the type of potassium channels expressed in the rat skeletal and cardiac muscles. J Biol Chem 266:13324-13328.
Mayhan W G. 2002. Cellular mechanisms by which tumor necrosis factoralpha produces disruption of the blood-brain barrier. Brain Res 927:144-152.
More et al. 2005. A novel Kv1.1 potassium channel blocking toxin from the venom of Palamneus gravimanus (Indian black scorpion), J. Venom. Anim. Toxins incl. Trop. Dis, 11(3).
Mourre et al. 1999. Distribution in rat brain of binding sites of kaliotoxin, a blocker of Kv1.1 and Kv1.3 alpha-subunits. J Pharmacol Exp Ther., 291(3):943-52.
Newsom-Davis et al. 1993. Immunological associations of acquired neuromyotonia (Isaacs' syndrome). Report of five cases and literature review. Brain 116 453-469.
Olamendi-Portugal et al. 1998. Two similar peptides from the venom of the scorpion Pandinus imperator, one highly effective blocker and the other inactive on K⁺ channels, Toxicon, 36:759-770.
Phipps et al. 1996. Chloride-channel block inhibits T lymphocyte activation and signalling. Cell Signal 8:141-149.
Rader et al. 1996. T cell activation is regulated by voltage-dependent and calcium-activated potassium channels. J Immunol 156:1425-1430.
Roberds et al. 1991. Cloning and tissue-specific expression of five voltage-gated potassium channel cDNAs expressed in rat heart. Proc Natl Acad Sci USA 88:1798-1802.
Rochat et al. 1998. Maurotoxin, a four disulfide bridges scorpion toxin acting on K⁺ channels, Toxicon, 36:1609-1611.
Ruiz-Arguelles et al. 2003. Antibody penetration into living cells: pathogenic, preventive and immunotherapeutic implications. Curr Pharm Des 9:1881-1887.
Rus et al. 2005. The voltage-gated potassium channel Kv1.3 is highly expressed on inflammatory infiltrates in multiple sclerosis brain. Proc Natl Acad Sci USA 102:11094-11099.
Rutecki P A. 1992. Neuronal excitability: voltage-dependent currents and synaptic transmission. J Clin Neurophysiol 9:195-211.
Siebenlist et al. 1994. Structure, regulation and function of NF-kappa B. Annu Rev Cell Biol 10:405-455.
Smart et al. 1998. Deletion of the K(V)1.1 potassium channel causes epilepsy in mice. Neuron 20:809-819.
Tsaur et al. 1992. Differential expression of K⁺ channel mRNAs in the rat brain and downregulation in the hippocampus following seizures. Neuron 8:1055-1067.
van Horssen et al. 2006. TNFα in cancer treatment: Molecular Insights, Antitumor Effects, and Clinical Utility. The Oncologist 11:397-408.
Verheugen et al. 1997. A charybdotoxin-insensitive conductance in human T lymphocytes: T cell membrane potential is set by distinct K⁺ channels. J Physiol (Lond) 503:317-331.
Vincent et al. 2004. Potassium channel antibody-associated encephalopathy: a potentially immunotherapy-responsive form of limbic encephalitis. Brain 127:701-712.
Vu et al. 2001. Differential involvement of initiator caspases in apoptotic volume decrease and potassium efflux during Fas- and UV-induced cell death. J Biol Chem 276:37602-37611.
Wallach et al. 1999. Tumor necrosis factor receptor and Fas signaling mechanisms. Annu Rev Immunol 17:331-367.
Wang et al. 1999. alpha subunit compositions of Kv1.1-containing K⁺ channel subtypes fractionated from rat brain using dendrotoxins. Eur J Biochem 263:230-237.
Zhou et al. 1998. Temperature-sensitive neuromuscular transmission in Kv1.1 null mice: role of potassium channels under the myelin sheath in young nerves. J Neurosci 18:7200-7215.

1. A method for causing synthesis and secretion of TNF-α by T-cells, comprising causing the selective blockage of the voltage-gated potassium channel Kv1.1 in the T-cells.

2. A method in accordance with claim 1, wherein said causing step comprises contacting the T-cells with a molecule causing the selective blockage of the Kv1.1 channel.

3. A method for treating a disease treatable by increased in vivo secretion of TNF-α, in a human or other animal subject, comprising causing T-cells to be present within the subject, which T-cells have a selective blockage of their voltage-gated potassium channel Kv1.1.

4. A method in accordance with claim 3, wherein the subject is one suffering from cancer, an immunodeficiency, or a deficiency in neuronal regeneration after neuronal injury or neurological disease.

5. A method in accordance with claim 4, wherein the subject is one suffering from locally advanced soft tissue sarcoma, metastatic melanoma, or another irresectable tumors of any histology, whereby amputation of a limb is to be avoided.

6. A method in accordance with claim 3, wherein said causing step comprises administering to the subject a molecule causing the selective blockage of the Kv1.1 channel.

7. A method in accordance with claim 3, wherein said causing step comprises removing autologous T-cells from the subject, contacting the autologous T-cells ex vivo with a molecule causing the selective blockage of the Kv1.1 channel, and administering the treated autologous T-cells back into the body of the subject.

8. A method in accordance with claim 7, wherein the subject is one suffering from locally advanced soft tissue sarcoma, metastatic melanoma, or another irresectable tumor of any histology, whereby amputation of a limb is to be avoided, wherein said treated autologous T-cells are administered by isolated limb perfusion setting.

9. A method in accordance with claim 7, wherein the subject is one suffering from liver cancer, whereby recruitment into the liver of various immune cells may be augmented, direct attack of the liver cancer by T-cell attack may take place, and direct eradication of the liver cancer by the TNFα secreted by the treated T-cells may take place.

10. A method for augmenting the permeability of the blood brain barrier and of the peripheral endothelium in a human or other animal subject in need thereof, comprising causing T-cells to be present within the subject, which T-cells have a selective blockage of their voltage-gated potassium channel Kv1.1.

11. A method in accordance with claim 10, wherein said causing step comprises administering to the subject a molecule causing the selective blockage of the Kv1.1 channel.

12. A method in accordance with claim 10, wherein said causing step comprises removing autologous T-cells from the subject, contacting the autologous T-cells ex vivo with a molecule causing the selective blockage of the Kv1.1 channel, and administering the treated autologous T-cells back into the body of the subject.

13. A method in accordance with claim 2, wherein said molecule causing the selective blockage of the Kv1.1 channel is Dendrotoxin-K.

14. A method in accordance with claim 2, wherein said molecule causing the selective blockage of the Kv1.1 channel is a specific anti Kv1.1 antibody.

15. A method in accordance with claim 2, wherein said molecule causing the selective blockage of the Kv1.1 channel is a molecule capable of selectively blocking the downstream pathways of the Kv1.1 channel.

16. A method for inhibiting the synthesis and secretion of TNF-α by T-cells, comprising causing the Kv1.1 channel to be opened or causing a blockade of the Kv1.1 channel to be overcome.

17. A method for treating a disease or condition treatable by decreased in vivo secretion of TNF-α, in a human or other animal subject, comprising causing the Kv1.1 channel on T-cells of the subject to be opened or causing a blockade of the Kv1.1 channel on T-cells of the subject to be overcome.

18. A method in accordance with claim 17, wherein the subject is one suffering from rheumatoid arthritis or post-trauma conditions.

19. A method in accordance with claim 17, wherein the disease or condition is PNH associated with Kv1 Abs; encephalitis associated with Kv1 Abs; and episodic-ataxia type 1 (EA-1) associated with different mutations of the gene that codes for the human Kv1.1 channel.
